(19) United States
(12) Patent Application Publication
(10) Pub. No.: US 2017/0175143 A1
(43) Pub. Date: Jun. 22, 2017

(54) METHOD FOR EDITING A GENETIC SEQUENCE

(71) Applicant: REGENTS OF THE UNIVERSITY OF MINNESOTA, Minneapolis, MN (US)

(72) Inventors: Jakub Tolar, Minneapolis, MN (US); Bruce Robert Blazar, Golden Valley, MN (US); Daniel Francis Voytas, Falcon Heights, MN (US); Mark John Osborn, Minneapolis, MN (US)

(21) Appl. No.: 15/311,685

(22) PCT Filed: May 20, 2015
(86) PCT No.: PCT/US15/31807

§ 371 Date: Nov. 16, 2016

Related U.S. Application Data
(60) Provisional application No. 62/000,590, filed on May 20, 2014.

(51) Int. Cl. C12N 15/90; C12N 5/0789; A61K 35/28; C12N 9/22

(52) U.S. Cl. CPC C12N 15/907; C12N 9/22; C12N 5/0647; A61K 35/28; A61K 2035/124

(57) ABSTRACT

This disclosure describes methods, polynucleotides, cells, compositions, and treatment methods that involve channeling a genomic nucleotide sequence. Generally, the method includes introducing a donor polynucleotide and a nucleotide that encodes an enzyme that cuts at least one strand of DNA into a cell that has a genomic sequence in need of editing, allowing the enzyme to cut at least one strand of the genomic sequence, and allowing the donor sequence to replace the genomic sequence in need of editing.

### CROSS-REFERENCE TO RELATED APPLICATION

This application claims priority to U.S. Provisional Patent Application Ser. No. 62/000,590, filed May 20, 2014, which is incorporated herein by reference.

### SUMMARY

This disclosure describes, in one aspect, a method for changing a genomic nucleotide sequence. Generally, the method includes introducing a donor polynucleotide and a nucleotide that encodes an enzyme that cuts at least one strand of DNA into a cell that has a genomic sequence in need of editing, allowing the enzyme to cut at least one strand of the genomic sequence, and allowing the donor sequence to replace the genomic sequence in need of editing.

In some embodiments, the genomic sequence can include a FANCC locus with a c.456+4A>T mutation or an equivalent thereof. In these embodiments, the donor polynucleotide can include a FANCC locus with a wild-type c.456+4A or an equivalent thereof so that the edited version of the sequence in need of editing includes the FANCC locus with a wild-type c.456+4A or an equivalent thereof.

In some embodiments, the enzyme can be a nuclease or a nickase.

In some embodiments, the donor polynucleotide can further include a selectable marker.

In some embodiments, the donor polynucleotide can further include at least one silent DNA polymorphism.

In some embodiments, the donor sequence replaces the genomic sequence in need of editing by homology-directed repair. In other embodiments, the donor sequence replaces the genomic sequence in need of editing by non-homologous end-joining.

In some embodiments, the cell is a pluripotent cell, a multipotent cell, a differentiated cell, or a stem cell. In some of these embodiments, the cell is homozygous for the c.456+4A>T mutation. In other embodiments, the cell may be a CD34+ human hematopoietic stem cell.

In another aspect, this disclosure describes an isolated cell prepared by any embodiment of the method summarized above.

In another aspect, this disclosure describes a population of cells prepared by any embodiment of the method summarized above.

In another aspect, this disclosure describes an expanded population of cells that are progeny of a cell prepared by any embodiment of the method summarized above.

In another aspect, this disclosure describes a polynucleotide that includes a promoter sequence, a polynucleotide encoding a functional portion of a Cas9 nuclease operably linked to the promoter sequence, and a polyadenylation signal operably linked to the polynucleotide encoding a functional portion of a Cas9 nuclease.

In another aspect, this disclosure describes a polynucleotide that includes a promoter sequence, a polynucleotide encoding a functional portion of a Cas9 nickase operably linked to the promoter sequence, and a polyadenylation signal operably linked to the polynucleotide encoding a functional portion of a Cas9 nickase.

In another aspect, this disclosure describes a method of treating a condition in a subject caused by a genetic mutation. Generally, the method includes the method comprising obtaining a plurality of pluripotent cells from the subject, introducing into at least one cell: a polynucleotide that encodes an enzyme that cuts at least one strand of DNA and a donor polynucleotide that encodes a version of the genomic sequence edited with respect to the genetic mutation, allowing the enzyme to cut at least one strand of the genomic sequence, allowing the donor sequence to replace the genomic sequence that includes the genetic mutation with the edited version, expanding the cell having the edited genomic sequence, and introducing a plurality of the expanded cells comprising the edited genomic sequence into the subject.

In some embodiments, the condition can be Fanconi's anemia. In some of these embodiments, the genomic sequence that includes a genetic mutation can be a FANCC locus with a wild-type c.456+4A>T mutation. In some of these embodiments, the donor polynucleotide can encode a FANCC locus with a wild-type c.456+4A.

In some embodiments, introducing the plurality of expanded cells into the subject results in correction of the FANCC locus.

In some embodiments, introducing the plurality of expanded cells into the subject results in restoration of proper splicing of FANCC mRNA.

In some embodiments, introducing the plurality of expanded cells into the subject results in phenotypic rescue of the subject.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. FANCC c.456+4A>T gene targeting. (A) FANCC locus with the c.456+4A>T mutation shown at the far right. The TALEN right and left array binding sites are underlined and the CRISPR gRNA recognition site is italicized. (B) TALEN repeat variable diresidue (RVD) base recognition and target site binding. The RVDs NN, NI, HD, and NG bind G, A, C, and T, respectively, and are reflected in the full sequence array below. The left and right TALEN arrays are linked to the nuclease domain of the FokI endonuclease that dimerize at the target and mediate cleavage of the DNA in the spacer region separating each array. (C) CRISPR architecture and FANCC gene target recognition. A gRNA chimeric RNA species has a gene-specific component (upper-case) that recognizes a 23 bp sequence in the FANCC gene (highlighted sequence) with the 3′ terminal NGG protospacer adjacent motif shown in red letters. The remainder of the gRNA (lower-case) are constant regions that contain secondary structure that interacts with the Streptococcus pyogenes Cas9 nuclease protein. The Cas9 RuvC-like and HNH-like domains mediate non-complementary and complementary DNA strand cleavage. A D10A mutation in the RuvC domain converts the complex to a nickase. (D) DNA expression platforms. Plasmid-encoded TALENs containing an N-terminal deletion of 152 residues of Xanthomonas TALENs, followed by the repeat domain, and a +63 C-terminal sub-region fused to the catalytic domain of the FokI nuclease under control of the mini-CAGGs promoter and the bovine growth hormone polyadenylation signal (pA). Cas9 nuclease or RuvC D10A nickase were expressed from a plasmid containing the CMV promoter, and bovine growth hormone pA. gRNA gene expression was mediated by the U6 polymerase III promoter and a transcriptional terminator (pT). (E) Nuclease activity assessment by the SURVEYOR assay (Transgenomic, Inc., Omaha, Nebr.). The FANCC locus in cells that received TALENs (nuclease target site indicated by left box), Cas9 nuclease, Cas9 nickase with corresponding gRNA (target site indicated by right box), or a GFP-treated control group (labeled ‘C’) were amplified with primers (arrows) yielding a 417 bp product. Nuclease or nickase generated insertions or deletions from NHEJ result in heteroduplex formation with unmodified amplicons that are cleaved by the mismatch dependent SURVEYOR nuclease. For TALENs these cleavage products are 277 bp and 140 bp and for CRISPR/Cas9 are 228 bp and 189 bp. SURVEYOR analysis of 293T cells (F) or FA-C fibroblasts (G). Equivalent amounts of DNA were amplified using the primers in 1(E) and showed post-Surveyor fragmentation patterns consistent with TALEN or CRISPR/Cas9 activity. Arrows indicate the cleavage bands. Data shown are representative gels of four experiments each. Mw=molecular weight standards, C=GFP-treated cells serving as the control. Gel exposure time for 293T cell Surveyor group was 750 milliseconds and 1.5 seconds for FA-C cells.

FIG. 2. Traffic light reporter assessment of DNA repair fates. (A) schematic of the TLR reporter. The FANCC CRISPR/Cas9 target sequence is contained within the dashed lines and was inserted into the GFP portion of the construct resulting in an out of frame GFP. The +3 picornaviral 2A sequence allows the downstream non-functional +3 mCherry to escape degradation of the non-functional GFP. Following target site cleavage in the presence of an exogenous GFP donor (box labeled ‘dsGFP donor’) the GFP gene is repaired by HDR and expresses GFP (+1 GFP), but not the inactive mCherry (+3 mCherry). DNA repair by NHEJ can result in a frameshift that restores the mCherry ORF (+1 mCherry) resulting in red fluorescence and inactive GFP (+3 GFP). (B-I) 293T TLR cell line treatment with CRISPR/Cas9 nuclease and nickases. A stable 293T cell line with an integrated copy of the FANCC TLR construct was generated that at its basal level was GFP negative and expressed <0.5% mCherry (B). (C) Donor only treated cells showing no endogenous HDR at donor concentrations of 250 ng (C i), 500 ng (C ii), or 1000 ng (C iii). (D, E) Representative FACs plots of FANCC-TLR-293T cell line transfected with the target gRNA and the Cas9 nuclease (D) or nickases (E) with GFP (x-axis) and mCherry (y-axis) measured at 72 hours post transfection. Panels i, ii, and, iii received 250, 500, or 1000 ng of the GFP donor, respectively. (F-H) Quantification of data shown and representation in graphical format from four independent experiments with the three different donor concentrations for the nuclease and nickases. The basal level of mCherry from 2(B) was subtracted from all treatment groups. For the nuclease p=<0.05 for NHEJ (mCherry) vs GFP (HDR). For the nickasesp=<0.05 for GFP (HDR) vs mCherry (NHEJ). (I) HDR ratio for nuclease and nickases. To determine the HDR ratio the percentage of cells expressing GFP was divided by those expressing mCherry at each donor concentration. Mean+/−sd are graphed and for nickases vs nuclease at 250 ng of donor, p=0.7, at 500 ng of donor p=0.04, and at 1000 ng of donor p=<0.001.

FIG. 3. Off-target sequence analysis. (A) In silico off-target site acquisition. The CRISPR Design Tool identified five intragenic off-target sites. Chromosomal location and gene name are shown with the FANCC target locus at top. Mismatches between FANCC target and off-target sites are underlined. (B) SURVEYOR nuclease assessment of off-target sites. Off-target alleles for 293T cells treated with nuclease (‘Nu’), nickase (‘Ni’) or GFP (‘G’) were amplified and assayed by the SURVEYOR procedure. Arrows indicate a cleavage product present in all three-treatment groups, which indicates the presence of a natural polymorphism. At right in 3(A) is the % modification (‘% Mod’) using the CRISPR nuclease (‘nuc’) or nickase (‘nick’) at each target site determined by SURVEYOR.

FIG. 4. (A) Integrase-deficient lentiviral gene tagging. (C) Diagram of self-inactivating integrase deficient GFP lentiviral cassette whose expression is regulated by the CMV promoter (sin.p11.CMV.GFP). In the presence of the TALEN or CRISPR/Cas9 that generate DNA DSBs or nicks a full copy of the viral cassette can be trapped at the on or off target break site where it remains permanently. (B) FACS analysis of IDLV treatment groups. Seven days post-IDLV treatment+/−concomitant nuclease and nickase delivery, the cells were assessed for GFP (labeled ‘7 days’). The sorted cell (Post sort′) populations were analyzed five days after the initial sort. (C) PCR screen for IDLV at FANCC and off-target sites. PCR assay using a 3′ LTR primer (right-pointing arrow) and a FANCC or OT locus-specific primer (left-pointing arrow) was performed. (D) FANCC locus-specific IDLV integration was observed and white arrows show amplicons that were sequenced. (E) Off-target IDLV screen. Cells from the CRISPR/Cas9 nuclease and nickase treatment groups were screened with an LTR forward and HERC2 (OT1), RLF (OT2), HNF4G (OT3), ERC2 (OT4), or LOC399715 (OT5) reverse primers.

FIG. 5. Unbiased genome wide screen for off-target loci. (A) Experimental workflow. Duplicate samples of 293T cells with integrated IDLV were subjected to nrLAM PCR and LAM PCR using MseI or MluCI enzymes and next generation sequencing with Illumina MiSeq deep sequencing. The data set was then refined using the High-Throughput Site Analysis Pipeline (HISAP). HISAP trims the sequence reads to remove vector and linker nucleotides in order to retain only the host genomic fragment amplicons. Redundant/identical sequences are consolidated and then mapped and annotated using the BLAT UCSC Genome Informatics database. The prevalence of CLIS in proximity to a locus is then assessed. (B) CLIS identification of IDLV integrants. The sample identifiers and number of sequence reads analyzed for each is indicated at left. The total number of IS for each sample is shown and the number of CLIS (X=no CLIS identified for IDLV only treatment group) observed. For all of the reagents the CLIS were localized only to the FANCC locus and were located within a 80 bp window.

FIG. 6. FANCC donor design and homology-directed repair. (A) The FANCC locus with the c.456+4A>T intronic mutation indicated with the downward arrow and asterisk. Left and right arrows indicate the endogenous genomic primers used for HDR screening. (B) Gene correction donor. The donor is shown in alignment relative to the endogenous locus. The plasmid donor contains a 1.3 kb left arm of homology that includes FANCC genomic sequences, silent mutations to prevent nuclease cutting of the donor, and the normalized base for the c.456+4A>T mutation (lightened region). Following this was a loxP flanked PGK promoter-regulated puromycin-T2A-FANCC expression cassette and a right donor arm that is 0.8 kb in length. Arrows show the donor specific PCR primers used for PCR analysis of CRISPR treated, selected, and expanded clones. (C) Representative gel image of PCR screening approach for the left (‘Lt’) and right (RV) HDR using the donor-specific and locus-specific primers from (A) and (B). (D) The number of gene corrected clones obtained. Numbers indicate the number of clonally expanded cells that showed a positive HDR PCR product. Two independent experiments were performed and the data is pooled together to obtain the total number of clones positive for HDR (E) HDR mediated c.456+4A>T mutation correction. Representative Sanger sequence data of the c.456+4A>T locus in untreated cells (top) and gene corrected clones (bottom). Shaded columns indicate the mutant thymine or corrected adenine base. Arrows on bottom sequence file shows the donor derived silent mutations present in the corrected clones.

FIG. 7. CRISPR-mediated restoration of FANCC. (A) The FANCC locus with mutation indicated with a red asterisk. The mutation results in aberrant splicing (top dashed line) that cause exon 4 (asterisk) skipping. Normal splicing is indicated by the bottom dashed lines. Third box represents exon 3, fourth box represents exon 4, fifth box represents exon 5, and the eighth box represents exon 8. (B) FANCC transcripts. The c.456+4A>T-mutation induced exon skipping results in deletion of exon 4. Gene correction results in restoration of exon 4 in the transcript. The right-pointing arrow indicates an allele specific primer for the silent base changes that were introduced by donor derived HDR. The left-pointing arrow represents an exon 8 specific primer. (C) Allele specific PCR of nickase and nuclease corrected cell clones. A representative gel of an allele specific PCR showing normalized transcripts in the nuclease and nickases clones. The specificity of the primer set is evident due to absence of amplification in FA-C(FC) or wildtype (WT) cells. To insure that cDNA was amplification grade, samples were subject to PCR with GAPDH primers (bottom). Mw=molecular weight standards. (D) Sanger sequencing of gene modified allele. At left is the start of exon 4 with arrows indicating the silent polymorphisms that were incorporated into the genome-targeting donor. At right is the junction (shaded column) of the restored exon 4 contiguous with exon 5. (E-F) FANCC protein activity. Graph is a representation of four experiments utilizing flow cytometric analysis of phosphorylated γ-H2AX in FA cells that are untreated or treated with 2 mM hydroxyurea. Nuclease or nickases clones were assessed simultaneously and data are presented as the mean fluorescence intensity (MFI) of the phospho-γ-H2AX antibody signal.

FIG. 8. CRISPR activity assessment in hematopoietic stem cells. (A) Purity and gene transfer. Human CD34+ HSCs were purified from total bone marrow and either left unstained or stained with an anti-CD34 antibody. Purified cells were transfected with a GFP plasmid (pmax-GFP) and fluorescence assessed at 48 hours. (B). CRISPR/Cas9 activity. Cas9 nickase or nuclease plasmid DNA with a plasmid encoding the gRNA were introduced into HSCs using the gene transfer conditions in (A). The Surveyor nuclease assay was performed on genomic DNA 72 h post gene transfer. Gel and FACs plots are representative of two independent experiments. Negative control (negC) was GFP treated HSCs. Positive control (posC) were 293 Ts treated with Cas9 nuclease.

FIG. 9. CRISPR NHEJ quantification. (A) mean fluorescence intensity of 293T or FA-C fibroblasts determined from four groups of cells co-transfected with an mCherry plasmid and the Cas9 nickase or nuclease with FANCC gRNA. The differences between nickases and nuclease treated cells was not statistically significant. (B) SURVEYOR assay. The gels in FIGS. 1(F) and 1(G) were overexposed for three seconds for 293T and FA-C cells to determine NHEJ rates of the nickases by densitometry post-SURVEYOR nuclease treatment. Arrows indicate the predicted size DNA fragments (C) Cas9 cleavage rates. Nuclease rates of cleavage were determined by densitometry from the gels in FIG. 1(F) and FIG. 1(G) with exposure times of 750 ms and 1500 ms for 293 Ts and FA-C fibroblasts, respectively. Nickase cleavage efficiencies were quantitated from the gels in (B). Nickase generated fragments were not visualized in FA-C cells. Values are from four individual experiments and are plotted as mean+/−s.d.

FIG. 10. IDLV LTR:FANCC junction PCR sequence. At the top, the sequences for the LTR forward primer (dotted underline) and for the FANCC genomic reverse primer (double underline) are shown. (A) CRISPR FANCC target site with protospacer adjacent motif (dashed underline). (B) Sequence of PCR product from IDLV and CRISPR nuclease-treated cells. LTR sequences are bolded; FANCC sequence is italicized. (C) Sanger sequence from nickase cells that received IDLV. The ‘i’ is the upper band from FIG. 2(D) and ‘ii’ is the lower band from FIG. 2(D). LTR and FANCC sequences are indicated as described above.

FIG. 11. Primary sequence data of HDR PCR assay. Top: A contiguous PCR amplicon derived from a locus-specific and donor primer set was sequenced and shows a seamless junction between the endogenous gene and the donor arm (marked with arrow). A distal silent polymorphism in the donor arm (box) was not incorporated, indicating crossing over from donor sequences proximal to the break site. Bottom: Shaded bases are donor-derived silent polymorphisms. Box indicates corrected base at the c.456+4A>T locus. ‘Query’ is the sequence derived from a CRISPR-corrected clone. ‘Sbjct’ is the reference donor sequence. Hatched lines indicate the intervening donor/PCR sequences that were deleted for clarity.

FIG. 12. FANCC c.456+4A>T cDNA sequencing. Primary sequence alignment of FANCC c.456+4A>T homozygous patient (top, ‘Query’) to a wild-type FANCC gene (bottom, ‘Sbjct’). Exon boundaries and deletion of exon 4 are shown. At bottom is the trace file from a sequencing reaction showing the exon 3:5 boundary.

FIG. 13. Gene-corrected c.456+4A>T cDNA sequencing. Primary sequence alignment of allele-specific PCR product FIG. 7(C). (top, ‘Query’) to a reference-predicted, donor-derived gene correction sequence (bottom, ‘Sbjct’). Shaded bases are silent polymorphisms unique to the donor.

FIG. 14. Exogenous donor sequence removal from nickases corrected clone by cre recombinase. Cre-recombinase was expressed in clones that underwent HDR. To confirm excision a FANCC locus PCR was performed that yielded two bands that were sequenced to show the recombined loxp sites (upper band/shading) representing the donor targeted allele and a lower band that was unmodified by the CRISPR/Cas9 (lower band/untargeted allele. Shading indicates the junction of the designed donor). Sequencing of the lower band in the nuclease treated clone revealed indels at the target site (data not shown).

### DETAILED DESCRIPTION OF ILLUSTRATIVE EMBODIMENTS

Genome engineering with designer nucleases is a rapidly progressing field, and the ability to correct human gene mutations in situ is highly desirable. Fibroblasts derived from a patient with Fanconi anemia (FA) were used as a model to test the ability and efficacy of the clustered regularly interspaced short palindromic repeats (CRISPR) Cas9 nuclease to mediate gene correction. The CRISPR/Cas9 nuclease and nickase each resulted in gene correction and, moreover, the nickase outperformed the nuclease in homology-directed repair (HDR). Homology-directed repair is a mechanism used by cells to repair double-stranded breaks in DNA using a homologous DNA sequence in the genome. Off-target effects were assessed suing, a predictive software platform to identify intragenic sequences of homology and a genome-wide screen using linear amplification mediated PCR (LAM-PCR). No off-target Cas9 activity was observed, showing that CRISPR/Cas9 candidate sites that possess sufficient sequence complexity function in a highly specific manner. These data show genome editing in FA, a DNA repair-deficient human disorder. These data are the first description of Cas9-mediated human disease gene correction.

The FANCC gene on chromosome 9 encodes a protein that is a constituent of an eight-protein Fanconi anemia core complex that functions as part of the Fanconi anemia pathway responsible for genome surveillance and repair of DNA damage. One cause of Fanconi anemia complementation group C (FA-C) is the c.456+4A>T (previously c.711+4A>T; IVS4+4A>T) point mutation that results in a cryptic splice site that causes aberrant splicing and the in-frame deletion of FANCC exon 4. The loss of exon 4 prevents FANCC participation in the formation of the core complex and results in a decrease in DNA repair ability. Typically, FA-C patients exhibit congenital skeletal abnormalities and progressive cytopenias culminating in bone marrow failure. Furthermore, FA-C patients exhibit a high incidence of hematological and solid tumors. People with Fanconi anemia who experience bone marrow failure, and for whom a suitable donor exists, are currently treated with allogeneic hematopoietic cell transplantation (HCT). However, risks associated with HCT provide an incentive to gene-correct autologous cells by gene addition or genome editing. Because of the pre-malignant phenotype Fanconi anemia patients possess, one consideration for any gene therapy is safety. The delivery of functional copies of the FANCC gene borne on integrating viral or non-viral vectors is associated with an increased risk of insertional mutagenesis. In contrast, this disclosure describes precise gene targeting achieved using genome-modifying proteins.

Efficient genome editing relies on engineered proteins that can be rapidly synthesized and targeted to a specific genomic locus. Candidates able to mediate genome modification include, for example, the zinc finger nucleases (ZFN), transcription activator-like effector nucleases (TALENs), and CRISPR/Cas9 nucleases. ZFNs and TALENs include DNA-binding elements that provide specificity and are tethered to the non-specific FokI nuclease domain. Dimerization of the complex at a genomic target site results in the generation of a double-stranded DNA break (DSB). The generation of ZFNs can be challenging and typically involves the acquisition of specialized starting materials and methodologies that somewhat limits their broader application.

In contrast, the starting materials to generate the multi-repeat TALEN complexes are publicly available, and assembly of this protein by this method is much simpler than those required for ZFNs.

The Streptococcus pyogenes CRISPR/Cas9 platform is also user-friendly and contains two components: the Cas9 nuclease and a guide RNA (gRNA). The gRNA is a short transcript that can be designed for a unique genomic locus possessing a GN₂₀GG sequence motif and that recruits the Cas9 protein to the target site, where the Cas9 induces a double-stranded DNA break. gRNAs direct Cas9 using complementarity between the 5′-most 20 nucleotides and the target site, which must have a protospacer adjacent motif (PAM) sequence of the form NGG.

In the description that follows and as an illustrative example, TALENs and CRISPR/Cas9 were used for FANCC gene targeting by homology-directed repair. This disclosure provides using TALENs and CRISPR/Cas9 nucleases to accomplish genomic editing of a model point mutation, FANCC c.456+4A>T and its use therapeutically. The CRISPR/Cas9 nuclease platform showed a higher rate of activity and allowed for precise c.456+4A>T mutation correction, resulting in the restoration of normal splicing and the presence of donor-derived exon 4 in FANCC cDNA.

### Gene-Editing Platform Architecture and Activity

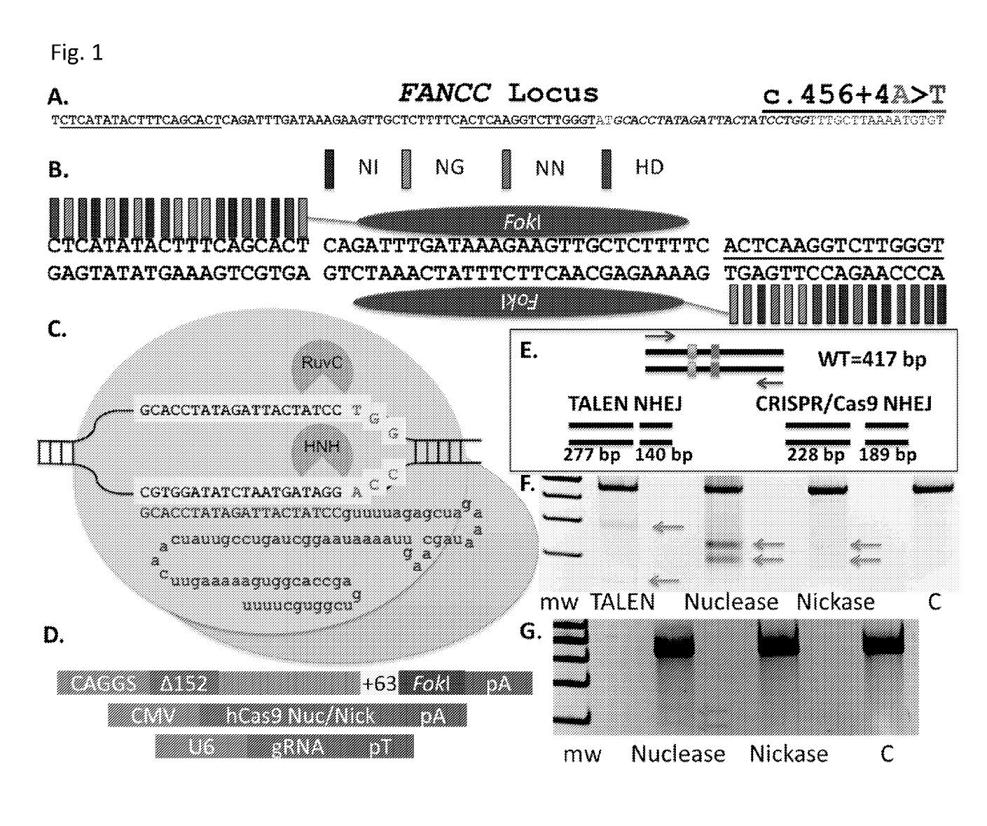

Using the TAL Effector-Nucleotide Targeter (Doyle et al., 2012, Nucleic acids research 40:W117-W122), Zinc Finger Targeter (Sander et al., 2007, Nucleic acids research 35:W599-W605; Sander et al., 2010, Nucleic acids research 38:W462-W468), and the CRISPR Design Tool (Hsu et al., 2013, Nature biotechnology 31:827-832), the FANCC gene sequence on chromosome 9 proximal to the c.456+4A>T locus was assessed for available nuclease target sites. No suitable ZFN sites were within 500 bp of the mutation, so a TALEN and CRISPR that were adjacent to one another and the c.456+4A>T site (FIG. 1(A)) were generated. TALENs include repeat units whose DNA recognition and binding ability are mediated by two hypervariable residues, are governed by a simple code, and are expressed as a fusion with the FokI nuclease domain that dimerizes at the target site (FIG. 1(B)). A CRISPR gRNA can contact the target locus and be recognized by a Cas9 protein that contains domains RuvC and HNH, each responsible for generating single-strand DNA breaks (nicks′) on opposite strands of the DNA helix (FIG. 1(C)). Inactivation of one of these domains converts Cas9 into a DNA nickase capable of cutting only one strand.

DNA expression constructs that included either a FANCC c.456+4A>T-specific TALEN, a FANCC c.456+4A>T-specific CRISPR nuclease, or a FANCC c.456+4A>T-specific CRISPR nickase (FIG. 1(D)) were delivered to 293T cells in order to assess rates of DNA-cutting in human cells using the SURVEYOR assay (Transgenomic, Inc., Omaha, Nebr.) that relies on non-homologous end joining (NHEJ)-mediated repair of nuclease-generated DNA lesions (Guschin et al., 2010, Methods in Molecular Biology 649:247-256).

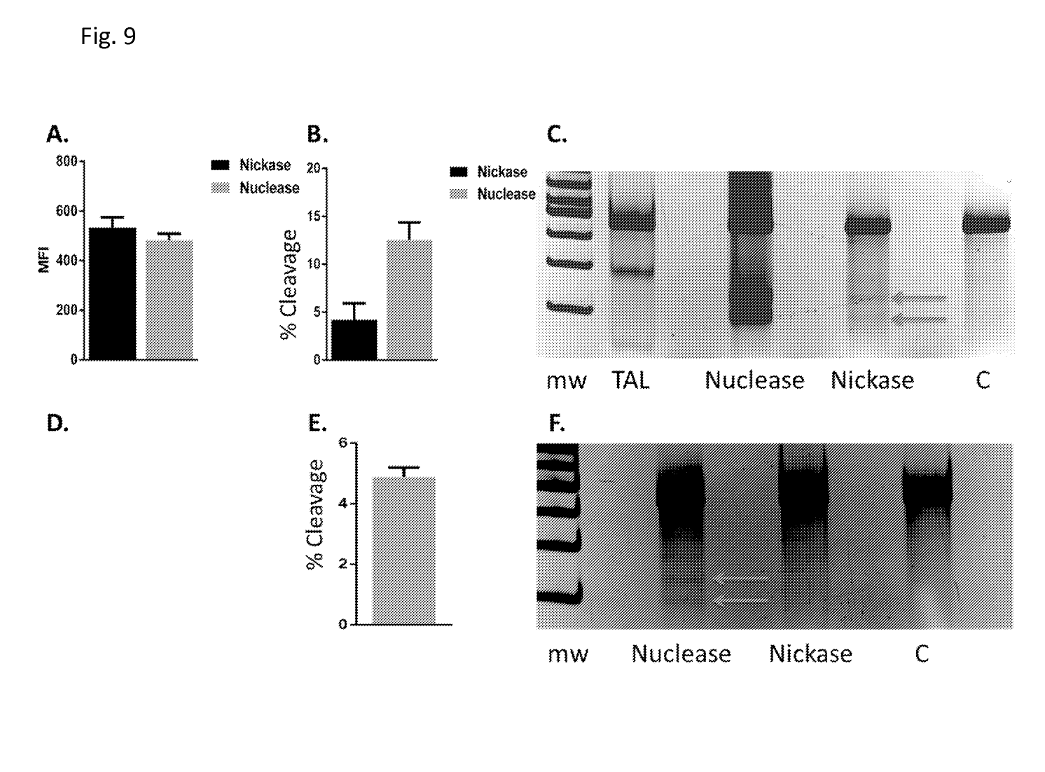

Densitometry analyses showed an approximately two-fold higher activity using the CRISPR/Cas9 nuclease, approximately 7% for TALEN and approximately 15% for CRISPR/Cas9 nuclease (FIG. 1(F) and FIG. 9). Because the CRISPR/Cas9 system exhibits a higher activation rate, the CRISPR/Cas9 system was used for determining activity rates in FA-C fibroblasts. Patient-derived cells showed editing rates of approximately 5% (FIG. 1(G) and FIG. 9). For both the 293T cells and FA-C fibroblasts, the nuclease version of Cas9 resulted in higher rates of activity compared to the nickases, using the SURVEYOR assay (Transgenomic, Inc., Omaha, Nebr.; FIGS. 1(F) and 1(G) and FIG. 9).

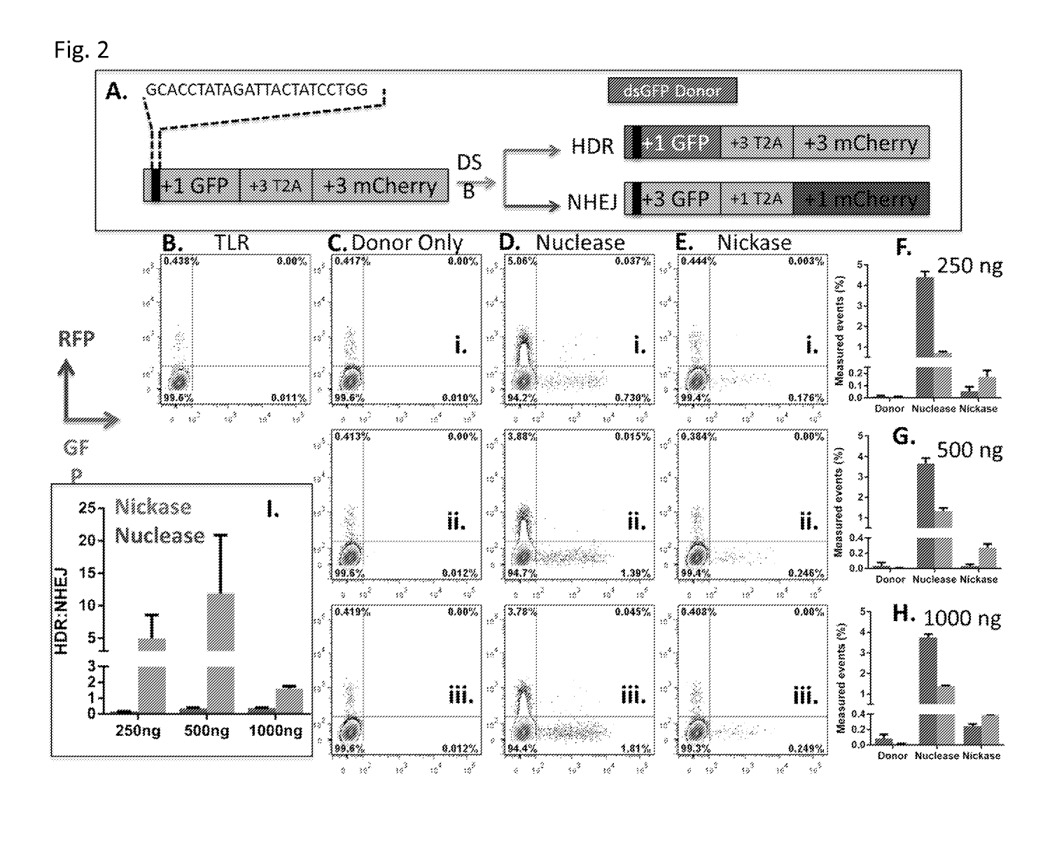

To insure that this differential activity profile was not due to unequal rates of gene transfer, an mCherry reporter was included at the time of transfection and the mean fluorescence intensity for each treatment group was nearly identical (FIG. 9). Thus, the use of nickases can promote a higher level of error-free HDR compared to NHEJ-mediated insertions/deletions. To definitively and quantitatively determine whether DNA nicking induced by the Cas9 D10A nickase resulted in preferential employment of the HDR arm of DNA repair, we used the Traffic Light Reporter (TLR) system (Certo et at, 2011, Nat. Methods 8:671-676) that allows for simultaneous quantification of NHEJ and HDR. This platform allows for a user-defined nuclease target sequence to be inserted into a portion of an inactive GFP coding region that is upstream of an out of frame mCherry cDNA (FIG. 2(A)). At its basal state the TLR construct does not express a functional fluorescent protein. Following cleavage of the target sequence, and in the context of an exogenous GFP donor repair template, however, GFP expression can be restored by HDR repair (FIG. 2(A)). Conversely, target site cleavage and repair by the error-prone NHEJ results in an in-frame mCherry (FIG. 2(A)). A 293T cell line with an integrated copy of the TLR containing the CRISPR/Cas9 FANCC target site was subsequently generated and used to assess rates of HDR and NHEJ for the nuclease and nickases versions of Cas9 using three different donor concentrations. The basal rates of green or red fluorescence for either untransfected or cells receiving the donor template only were minute (FIG. 2(B) and FIG. 2(C)). Nuclease delivery resulted in substantial rates of both mCherry and GFP fluorescence, showing that both mutagenic NHEJ and error free HDR can occur in response to a DSB (FIG. 2(D)). In contrast, a single stranded nick mediated by the D10A Cas9 nickase resulted in minimal levels of NHEJ induced red fluorescence with a preference toward HDR (FIG. 2(E)). In aggregate, over three doses of donor concentration, the nuclease mediated the highest levels of GFP by HDR. There was, however, a concomitant increase in NHEJ induced mCherry (FIG. 2(F)-(H)). The nickases showed lower overall rates of HDR compared to the nuclease. There was, however, minimal NHEJ (FIG. 2(F)-(H)). When these data are expressed as a ratio of HDR to NHEJ, the repair of DNA nicks shows a clear preference for HDR (FIG. 2(I)). These data show that the nickase version of Cas9 promotes HDR and minimizes NHEJ.

CRISPR on-Target and Off-Target Analysis

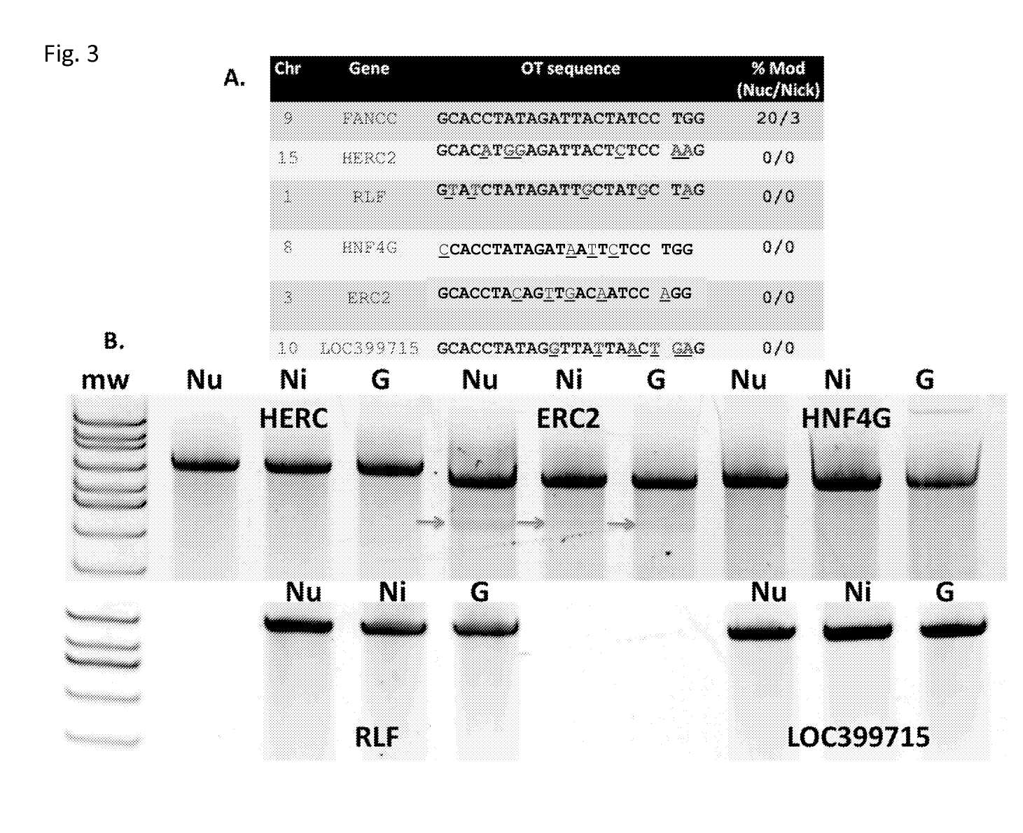

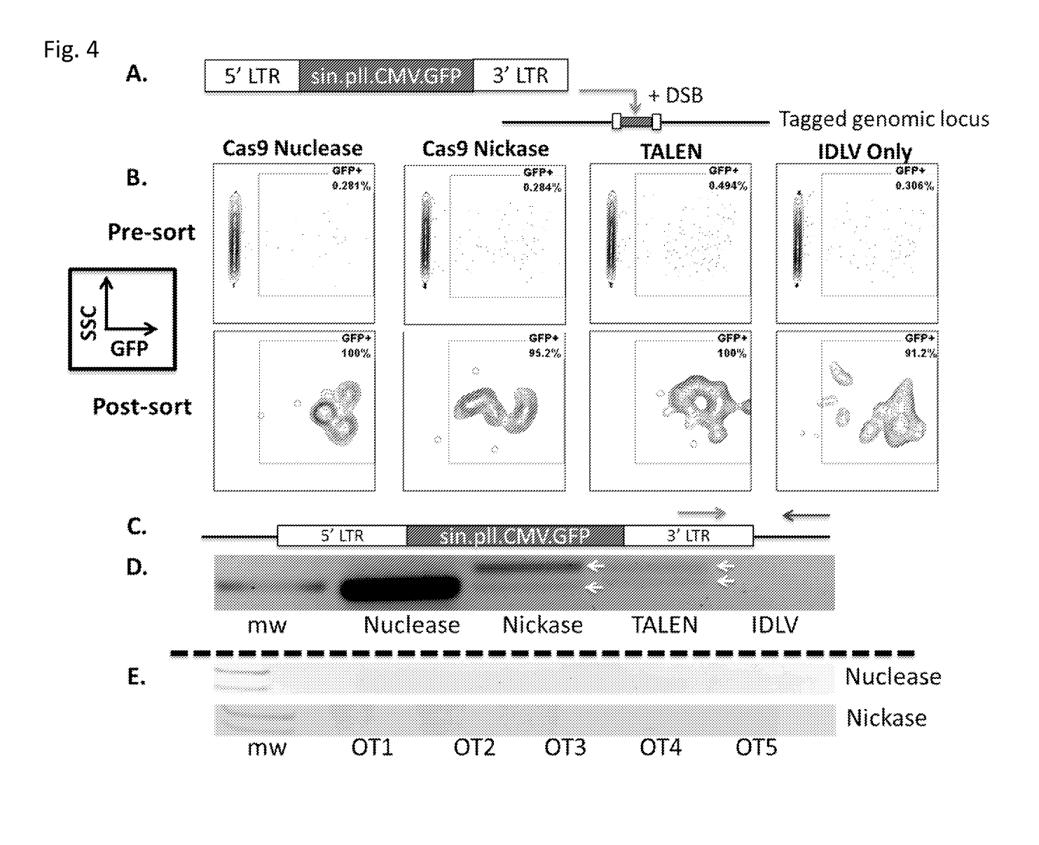

One factor in gene editing-based correction strategies is the potential for off-target effects due to sequence homology between the target site and non-target genomic loci. Therefore, the safety profile of the CRISPR/Cas9 reagent was assessed. CRISPR Design Tool (DNA2.0, Inc., Menlo Park, Calif.) analysis software can predict off-target sites and revealed five such sites within non-target locations for our FANCC CRISPR construct (FIG. 3(A)). To rigorously assess whether the FANCC CRISPR/Cas9 nuclease and nickase exhibited intragenic off-target activity, the SURVEYOR assay and an integrase-deficient lentiviral (IDLV) reporter gene trapping technique (Gabriel et al., 2011, Nature biotechnology 29:816-823) were used. SURVEYOR analysis (Transgenomic, Inc., Omaha, Nebr.) showed no demonstrable activity for the nuclease or nickase at the predicted intragenic off-target sites (FIG. 3(B)). Because the limit of detection with the SURVEYOR methodology has been reported to be approximately 1%, off-target effects were further assessed using tandem delivery of either the CRISPR/Cas9 nuclease or nickase with a green fluorescent protein (GFP) IDLV using a PCR-based gene trapping approach in order to maximize sensitivity (FIG. 4(A)). IDLV transduction of 293T cells resulted in approximately 80% GFP expression at 48 hours that rapidly diminished due to loss of episomal vector genomes during cell division, resulting in a low level of GFP cells that were then sorted to purity and expanded (FIG. 4(B)).

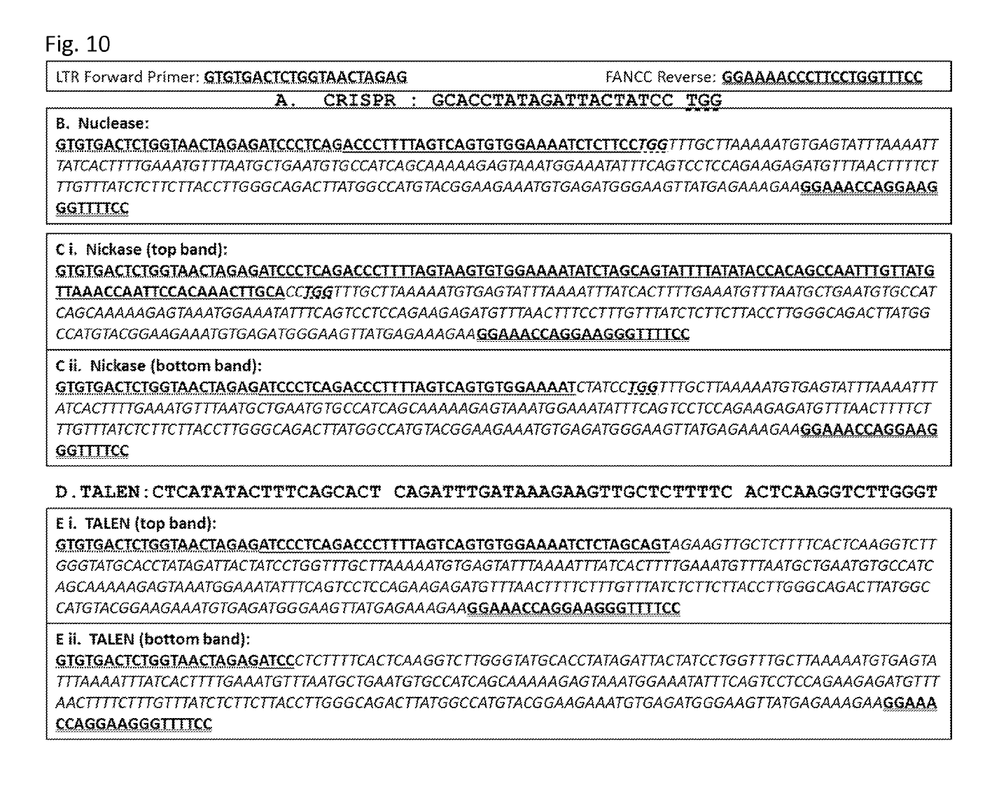

PCR analysis using a 3′ long terminal repeat (LTR) forward primer and a FANCC reverse primer (FIG. 4(C)) yielded a PCR product for the nuclease-treated cells and the nickase-treated cells but not IDLV-only control cells (FIG. 4(D)). Sequencing of these products showed an LTR:FANCC genomic junction immediately upstream of the CRISPR protospacer adjacent motif or the TALEN spacer, respectively (FIG. 10). These results show that the delivery of a GFP IDLV into cells results in trapping of the viral cargo at the site of a double-stranded DNA break, and they validate the methodology as a means to detect loci at which the nuclease is active. The use of an LTR primer and an off-target locus-specific primer failed to generate a product at any of the five off-target sites in CRISPR nuclease-treated or nickase-treated cells (FIG. 4(E)). In totality, these data suggest a favorable safety profile for our FANCC Cas9 nuclease and nickase, one that can provide precision gene editing with limited endogenous off-target gene disruption.

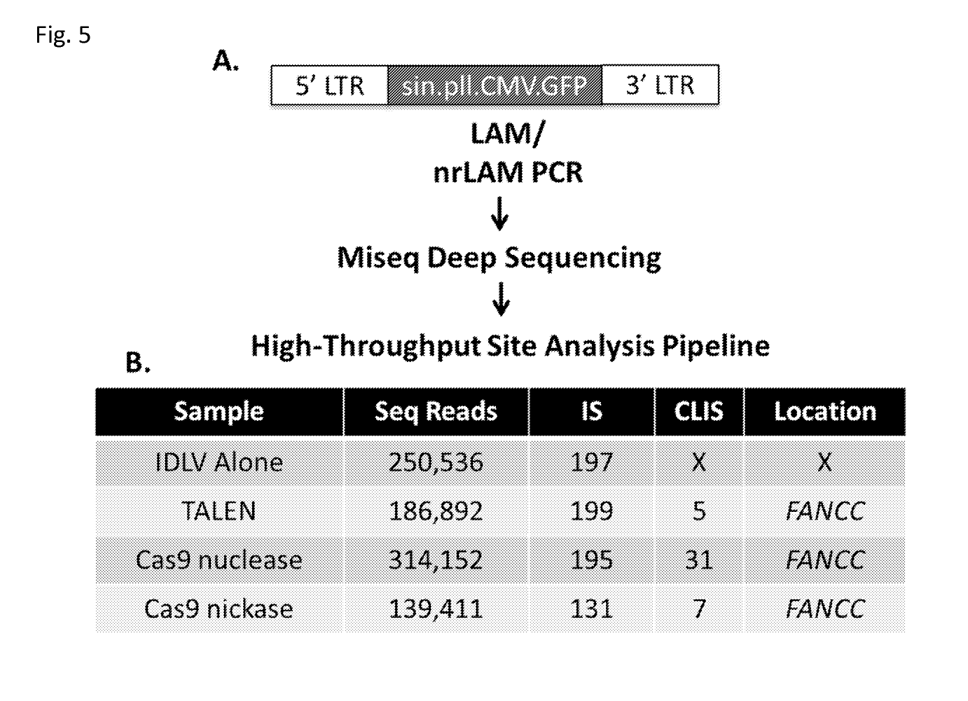

These analyses were biased toward specific loci predicted in silico. In order to fully evaluate the safety profile of these reagents, an unbiased, genome wide screen was performed. To identify the sites of integration of the IDLV, the samples were tested using LAM PCR and by nonrestrictive (nr)LAM PCR that is not reliant on a nearby restriction endonuclease site. The workflow and analysis is summarized in FIG. 5(A) and deep sequencing resulted in approximately 3.9 million individual paired sequencing reads with nearly 900,000 that could be mapped to the human genome using the high-throughput insertion site analysis pipeline. Within this, integration sites (IS) were identified with an IS being classified as a junction between viral LTR and a genomic sequence. Each treatment group contained between 130-200 IS (FIG. 5(B)) that could be further analyzed for the formation of clusters of integrations (CLIS). CLIS are defined as a minimum of two integration events within a ˜500 bp range of genomic DNA. Such limited range clusters in this context are considered to occur due to the recognition of a target DNA sequence (on or off target) by the gene editing reagent with subsequent DNA cutting and IDLV trapping. Further, the human genome was screened for CLIS at putative OT binding sites that contained up to 5 or 15 mismatches between the OT locus and FANCC target for CRISPR and TALEN, respectively. The results documented CLIS frequencies of 5-31 at the intended target site, while no CLIS were recovered at loci containing partial target site homology (FIG. 5(B)). Cumulatively, the data show highly specific CRISPR/Cas9 and TALEN reagents and support their application for precision gene editing approaches.

### Homology-Directed Repair

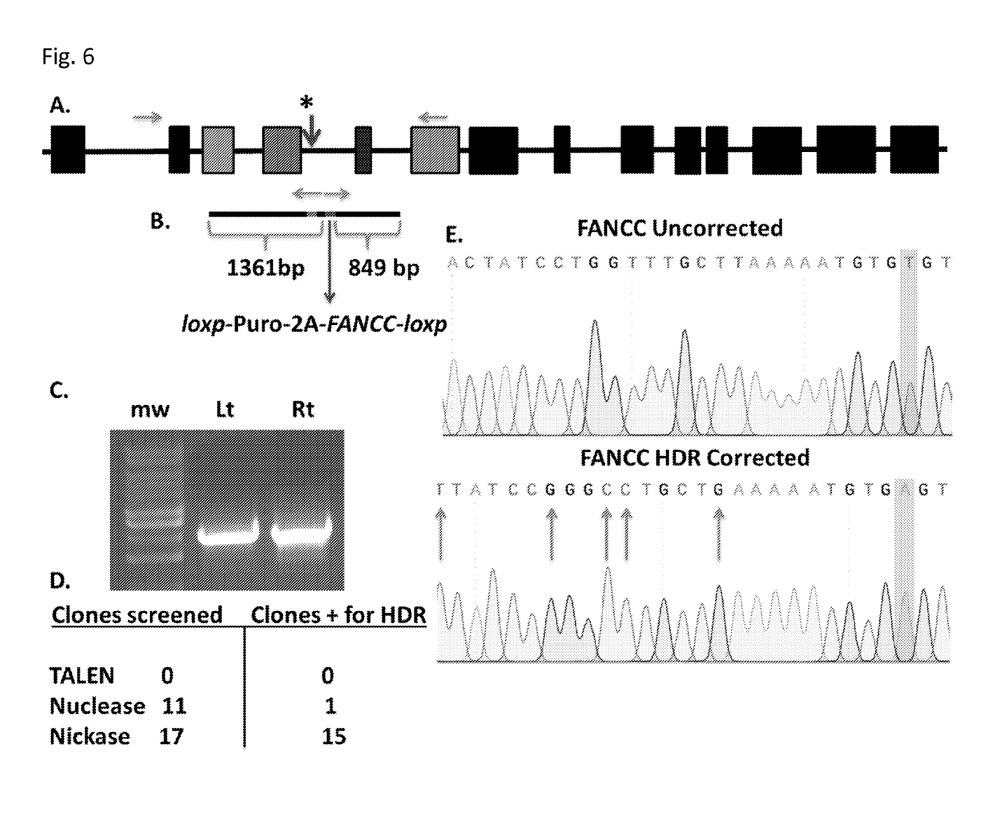

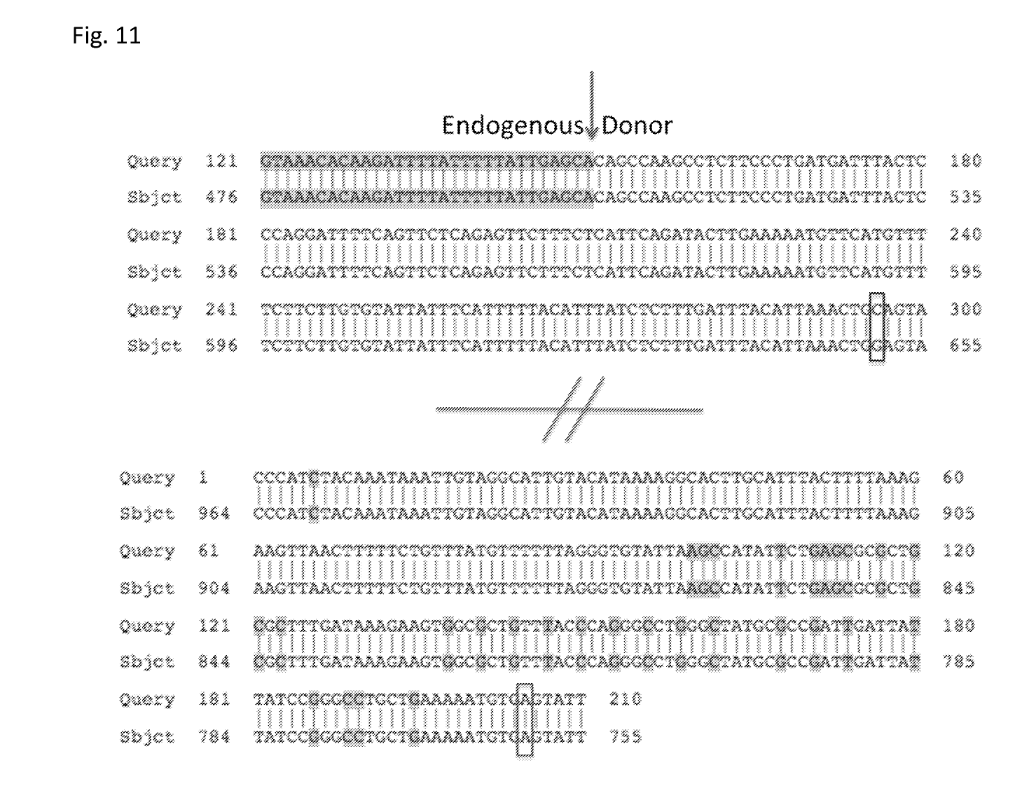

To test the ability of CRISPR/Cas9 to mediate FANCC gene homology-directed repair (HDR), a transformed skin fibroblast culture was derived from a FA-C patient homozygous for the c.456+4A>T mutation and treated the fibroblasts with the TALENs or the CRISPR/Cas9 genome editing reagents and a donor plasmid. The donor plasmid functions as the repair template following the generation of a double-stranded DNA break and spans a region of the FANCC gene from the third exon to the fifth intron (FIG. 6(A) and 6(B)). It also contains a selectable marker, as well as silent DNA polymorphisms designed to allow tracking of HDR events and to prevent FANCC-specific nuclease cutting of the FANCC donor sequences (FIG. 6(A) and 6(B) and SEQ ID NO:1). In bulk populations of cells, homology-directed repair was evident for the CRISPR/Cas9 nuclease and nickase as determined by PCR using donor-specific primers and locus-specific primers outside the donor arms (FIG. 6(A)-(C)). This bulk population was then plated at low density in order to isolate and expand single cell-derived clones. The CRISPR/Cas9 nuclease and nickase each resulted in numerous cell clones that showed evidence of homology-directed repair, with the nickase treatment resulting in the most clones exhibiting donor-derived repair (FIG. 6(D)). Sanger sequencing of the FANCC locus showed the presence of donor-derived polymorphisms as well as correction of the c.456+4A>T mutation (FIG. 6(E) and FIG. 11). These data document the ability of CRISPR/Cas9 reagents to mediate precise correction of FANCC c.456+4A>T mutation by HDR.

### Restoration of FANCC Gene Expression

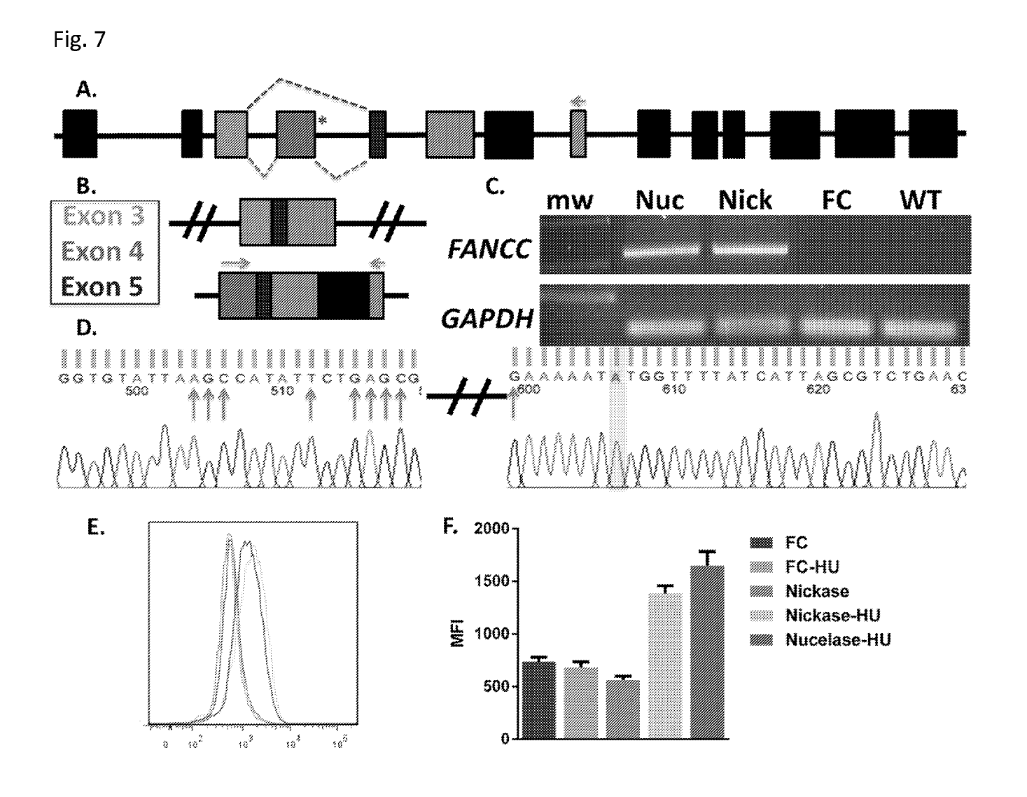

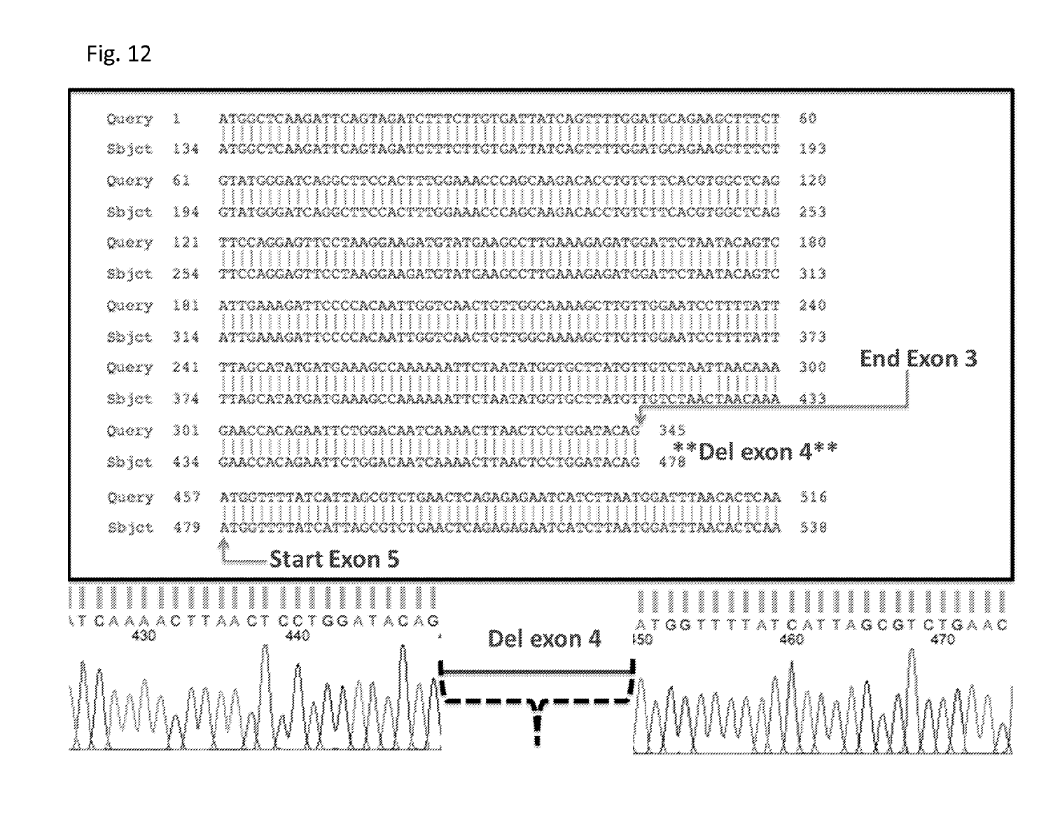

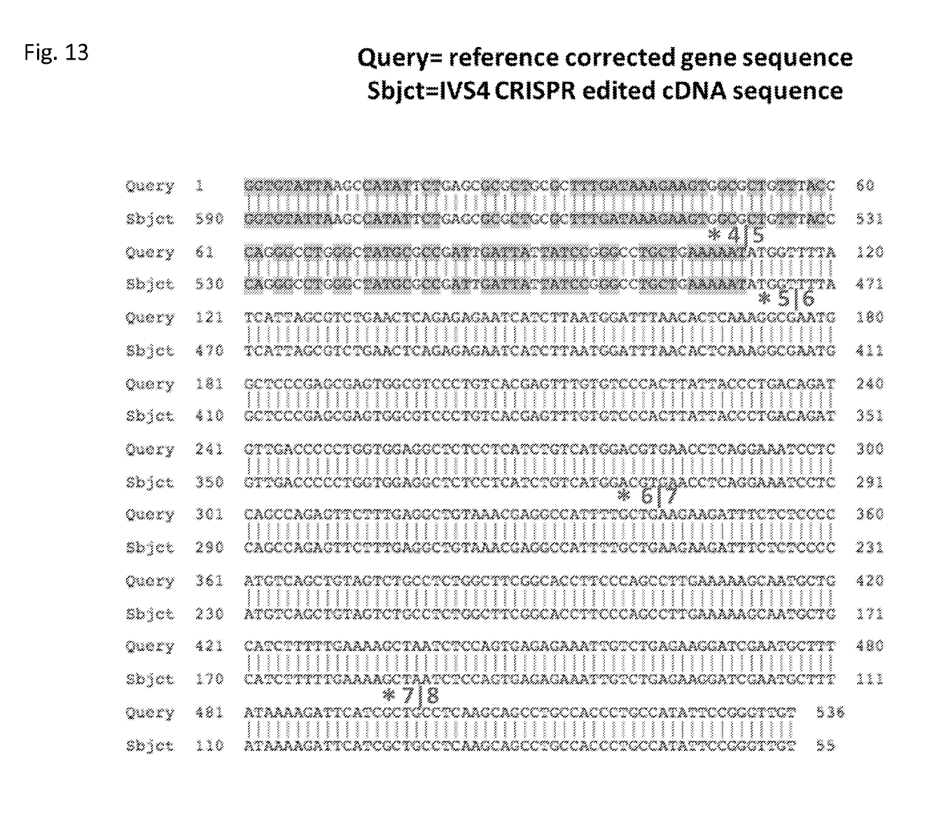

The result of the c.456+4A>T mutation is the skipping of exon 4 (FIG. 7(A) and 7(B) and FIG. 12). To determine whether genome editing by CRISPR/Cas9 resulted in restoration of exon 4 expression, corrected transcript-specific RT-PCR was performed using a forward primer that recognizes unique donor-derived bases and using a reverse primer in exon 8 that is several kilobases downstream of the terminus of the donor arm (FIGS. 7 (B) and 7(C)). CRISPR/Cas9 nuclease and nickase cells each showed the presence of the modified transcript, while untreated FA-C and wild-type cells did not show a product, thus confirming the specificity of the assay (FIG. 7(C)). To conclusively demonstrate seamless continuity of exon 4 with downstream exons, we sequenced the amplicons and showed the presence of polymorphisms present from homology-directed repair and intact exon:exon junctions (FIG. 7(D) and FIG. 13). These data confirm the ability of CRISPR/Cas9-mediated homology-directed repair of the c.456+4A>T mutation to restore proper expression of FANCC exon 4 in cells from an individual with FA-C.

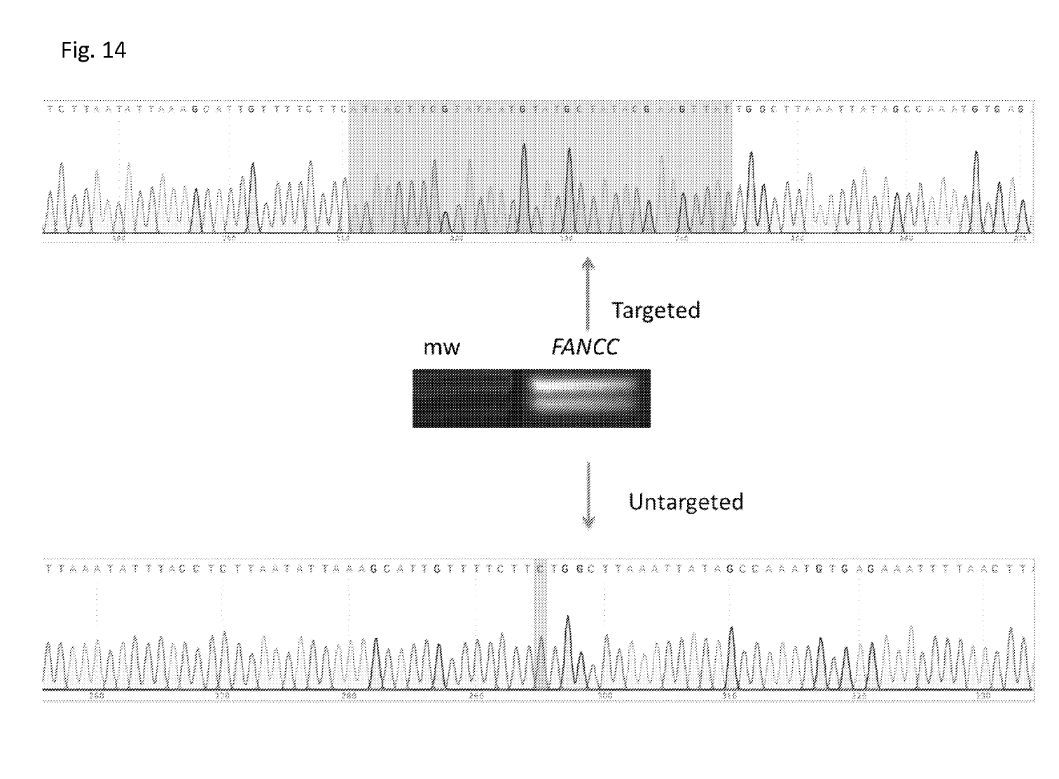

The positioning of our exogenous sequences (i.e., puromycin and FANCC cDNA) within the donor construct resulted in their insertion by homology-directed repair into an intronic sequence approximately 400 bp away from an exon and thus did not result in perturbation of splicing. However, to assess whether the gene correction observed at the DNA and mRNA levels extended to functional rescue, cre-recombinase was used to remove the floxed sequences (FIG. 14). Untreated FA-C cells do not phosphorylate γ-H2AX (FIG. 7(E)). The clones that were corrected by the nickase or the nuclease showed restored ability to phosphorylate γ-H2AX (FIG. 7(E)). In totality, the data show correction of the c.456+4A>T mutation in fibroblasts at the DNA, mRNA, and protein levels.

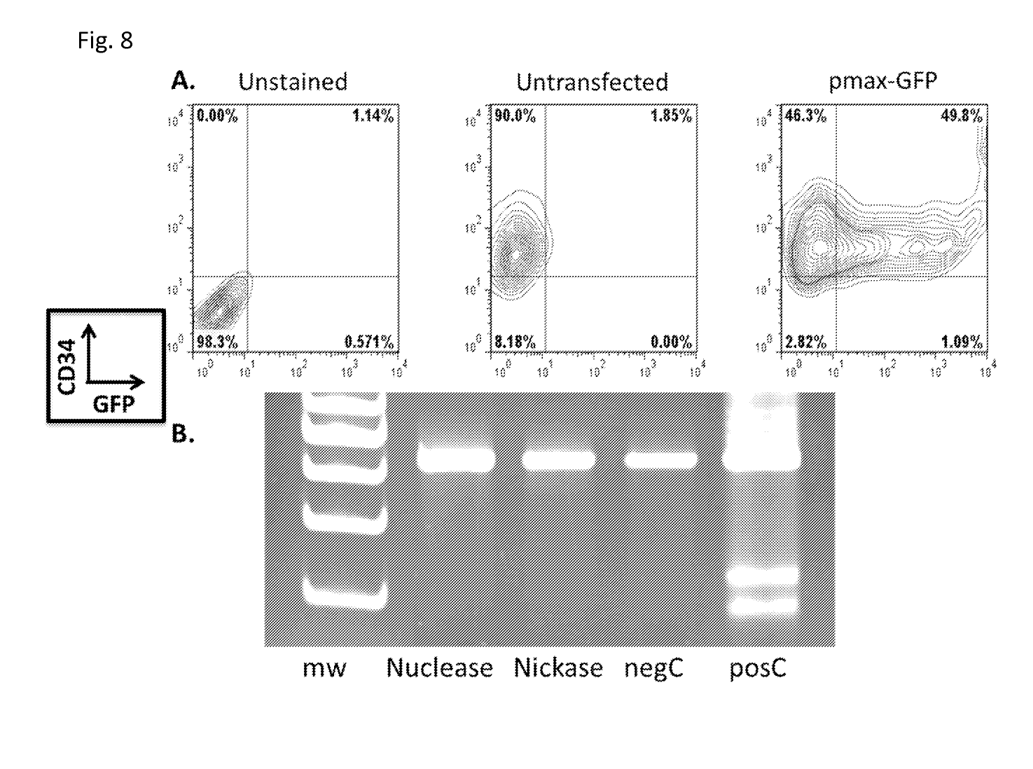

To extend these studies to therapeutic applications, the gene editing rates of the CRISPR/Cas9 reagents in CD34+ human hematopoietic stem cells were investigated. Using a highly pure population of hematopoietic stem cells (HSCs), electroporation-based delivery of a GFP plasmid DNA species was delivered at a rate of approximately 50% (FIG. 8(A)). Using these conditions, Cas9 nickase or nuclease plasmid with a FANCC gRNA plasmid were introduced and activity was assessed using the SURVEYOR method (Transgenomic, Inc., Omaha, Nebr.). These data showed no demonstrable activity at the FANCC locus in HSCs (FIG. 8(B)).

Thus, this disclosure describes TALEN and CRISPR/Cas9 genome editing systems for the FANCC locus as an exemplary model locus, observed higher activity rates using the CRISPR/Cas9 system (FIG. 1), and pursued its use for repair of the FANCC c.456+4A>T mutation. The CRISPR/Cas9 nuclease and nickases embodiments exhibited differing abilities of the Cas9 variants to mediate homology-directed repair of the mutation in patient-derived transformed fibroblasts using a donor that contained a floxed puromycin and FANCC cDNA flanked by arms of homology to the FANCC locus (FIG. 6(B)). Gene correction with high frequency was achieved using the D10A nickases (FIG. 6(D)). This resulted in restoration of proper splicing and functional rescue of the FA phenotype (FIG. 6 and FIG. 7).

The traffic light reporter system (Certo et al., 2011, Nat. Methods 8:671-676) was used to assess the preferred pathway of DNA repair for the CRISPR/Cas9 system. Directly comparing the two version of Cas9 showed that the HDR rates for the nuclease were higher than the nickase (FIG. 2(B)-(H)). However, this was offset by a high rate of nuclease-induced NHEJ that was essentially absent from nickases treated cells (FIG. 2). As such, expressing the outcome of DNA cleavage as a ratio of HDR versus NHEJ showed that the nickases possess a strong bias toward faithful gene repair by HDR (FIG. 2(I)). The phenotype of FA may make nickases especially valuable since DNA nicks can be resolved by an alternative HDR (altHDR) pathway that proceeds when BRCA2 or RAD51 are downregulated. Given the intimate connection of FANCC and other FA proteins with BRCA2 and RAD51 for mediating HDR following a DSB, FA cells may preferentially employ altHDR. Also, targeting the non-template strand, as described herein, can promote higher levels of HDR. The results further show that in FA nickases promote HDR and minimize NHEJ (FIG. 2, FIG. 6, and FIG. 7). This resulted in correction at the genomic locus, restoration of proper mRNA splicing, and phenotypic rescue in patient derived fibroblasts (FIG. 6 and FIG. 7).

One utility of the fibroblasts in the study described herein was to establish the possibility of using gene editing as a treatment option for the FA class of disorders. For many disorders, hematopoietic stem cells (HSCs) may represent a model cell type for precision gene targeting. FA may be uniquely suited to this approach, as bone marrow cells possess sensitivity to mytomycin C (MMC) where fibroblasts do not. As such, a selective advantage appears to exist in gene-corrected HSCs in FA. Thus, the this disclosure has established for the ability of CRISPR/Cas9 to mediate a gene correction event in FA can be enhanced with, for example, optimized HSC culture, expansion, and gene transfer as part of the formation of next generation therapies.

A second consideration for clinical use of gene editing reagents is a detailed analysis of off-target effects. In silico analysis identified five off-target sites within coding regions that shared significant sequence homology to the FANCC target site (FIG. 3(A)): HERC2 encodes a large protein believed to function as a ubiquitin ligase, RLF and HNF4G are predicted to be transcriptional regulators, ERC2 is involved in neurotransmitter release, and LOC399715 is an uncharacterized RNA gene. These sites were evaluated for evidence of CRISPR off-target activity by the SURVEYOR method (Transgenomic, Inc., Omaha, Nebr.) as well as a gene-trapping method. None of the off-target sites exhibited activity by SURVEYOR assay or by capture and detection of the IDLV cargo by a sensitive PCR-based assay (FIG. 3 and FIG. 4). The ability of IDLV to be trapped at both double and single stranded breaks provided a platform for the ultrasensitive, unbiased, genome-wide LAM PCR methodology to be employed to further assess off-target effects. Greater than 100,000 sequence reads were evaluable for each of the reagents and control samples (FIG. 5(B)). The numerous IS observed in control and TALEN or CRISPR/Cas9 treated cells show IDLV capture at genomic fragile spots that occur endogenously and independent of nuclease activity. Only cells treated with TALEN or Cas9 contained IDLV CLIS indicative of site-specific nuclease activity. The CLIS were solely localized to the FANCC locus showing that the reagents employed in our study are very specific. CRISPR/Cas9 specificity has conventionally been a concern when using a CRISPR/Cas9 system for genome editing. This concern has been overcome by rigorously designing CRISPR/Cas9 candidates to possess sufficient sequence complexity to minimize off-target effects. Doing so, as evidenced by the genome wide screen described herein, can result in a highly specific gene-editing reagent.

The cell type used for the off-target effects was carefully considered. 293T cells were used because their rapid proliferation would facilitate dilution of episomal IDLV, thus decreasing background and minimizing the number of ectopic IDLV integration events at genomic fragile sites. The 293T cells rapidly diluted the unintegrated IDLV (FIG. 4(B)). Due to the open chromatin profile of 293T cells, off-target events would manifest to the highest possible degree, thereby representing the most thorough and stringent screening procedure. Moreover, laboratory cell lines employed for IDLV gene mapping prove a useful predictor for gene editing off-target site analysis in primary cells. As such, the lack of off-target sites in 293 Ts suggests a highly specific reagent.

In summary, this disclosure shows that both the CRISPR/Cas9 nuclease-mediated and nickase-mediated direct c.456+4A>T mutation repair resulted in normalization of the FANCC transcript. The nickase-mediated mutation repair, in particular, was more efficient. Further, we provide support for a favorable safety profile using these synthetic molecules for correcting genetic disease in human cells. The observation that CRISPR/Cas9 mediates homology-directed repair in Fanconi anemia establishes proof of principle for the application of genome editing for human genetic disorders, including those with defects in the DNA repair pathway.

While described above in the context of repairing the c.456+4A>T mutation associated with Fanconi anemia, the methods described herein may be used to edit genomic sequences in any suitable manner. For example, the donor sequence may be designed to repair other point mutations, addition mutations, deletion mutations, or substitution mutations associated with conditions other than Fanconi anemia. As another examples, the methods may be used to introduce a nucleotide sequence associated with a desired phenotype, regulate expression of a gene by altering epigenetic architecture or binding of activating or repressing factors in the promoter/enhancer regulatory region, and/or multiplex these functions to turn on or off coding and regulatory nucleic acids (DNA or RNA). In short, the methods may be used to deliver any desired donor polynucleotide into a genomic sequence and to enable regulation of gene expression in sequence-specific fashion.

### DEFINITIONS

As used herein, the term “animal” refers to living multi-cellular vertebrate organisms, a category that includes, for example, mammals and birds. The term “mammal” includes both human and non-human mammals. Non-limiting examples of such include humans, non-human primates, dogs, cats, sheep, mice, horses, and cows. In some embodiments, the mammal is a human

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the terms “nucleic acid sequence,” “oligonucleotide,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. A polynucleotide can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can include modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

The term “encode” as it is applied to nucleic acid sequences refers to a polynucleotide that is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

Unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may include two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, a “pluripotent cell” also termed a “stem cell” defines a cell that can give rise to at least two distinct (genotypically and/or phenotypically) differentiated progeny cells and is less differentiated than the progeny cells. In another aspect, a “pluripotent cell” includes an Induced Pluripotent Stem Cell (iPSC), which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e., Oct-3/4; the family of Sox genes, i.e., Sox1, Sox2, Sox3, Sox 15 and Sox 18; the family of Klf genes, i.e., Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007. A “multi-lineage stem cell” or “multipotent stem cell” refers to a stem cell that reproduces itself and at least two further differentiated progeny cells from distinct developmental lineages. The lineages can be from the same germ layer (i.e., mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin). A “stem cell” may be categorized as somatic (adult) or embryonic. A somatic stem cell is an undifferentiated cell found in a differentiated tissue that can renew itself (i.e., is clonal) and, with certain limitations, can differentiate to yield each of the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. A clone is a line of cells that is genetically identical to the originating cell; in this case, a stem cell. Certain stem cells may be CD34+ stem cells. CD34 is a cell surface marker. An amino acid sequence for CD34 and a polynucleotide that encodes CD34 is reported under GenBank number M81104 (X60172).

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids in a protein peptide. As used herein the term “amino acid” refers to a natural, an unnatural amino acid or a synthetic amino acid, including glycine and both the D and L optical isomers, amino acid analogs and peptidomimetics.

The term “expanded” refers to any proliferation or division of cells. A “cultured” cell is a cell that has been separated from its native environment and propagated under specific, pre-defined conditions. The term “culturing” refers to the in vitro propagation of cells or organisms on or in media of various kinds. The descendants of a cell grown in culture may not be completely identical (i.e., morphologically, genetically, or phenotypically) to the parent cell. The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type.

As used herein, “treating” or “treatment” of a condition in a subject refers to reducing, limiting progression, ameliorating, or resolving, to any extent, the symptoms or signs related to a condition. “Symptom” refers to any subjective evidence of disease or of a patient's condition. “Sign” or “clinical sign” refers to an objective physical finding relating to a particular condition capable of being found by one other than the patient. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject—e.g., to a subject “at risk” of developing the condition. A subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

Unless defined otherwise, all technical and scientific terms used herein have the same meanings as commonly understood by one of ordinary skill in the art to which this technology belongs. Although exemplary methods, devices and materials are described herein, any methods and materials similar or equivalent to those expressly described herein can be used in the practice or testing of the present technology. For example, the reagents described herein are merely exemplary and that equivalents of such are known in the art.

The practice of the present technology can employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### EXAMPLES

### Research Subject Cell Line Generation and Culture

Informed consent was obtained from the parents of a child possessing the c.456+4A>T mutation and a skin punch biopsy was performed in accordance with the University of Minnesota Institutional Review Board requirements for research on human subjects. A fibroblast cell line was derived by dicing the skin tissue, covering it with a microscope slide, and adding complete DMEM (20% FBS, 100 U/mL nonessential amino acids, 0.1 mg/ml each of penicillin and streptomycin, and EGF and FGF at a concentration of 10 ng/mL) with culture under hypoxic conditions. A TERT-GFP lentiviral construct was then added to the cells.

### TALEN, CRISPR, and Donor Construction

The TALEN was constructed using the Golden Gate Assembly method and cloned into a CAGGs promoter-driven, homodimeric FokI endonuclease expression cassette (Cermak et al., 2011, Nucleic acids research 39(12):e82; Christian et al., 2010, Genetics 186(2):757-761). The Cas9 and Cas9 D10A plasmids were obtained from Addgene (Cambridge, Mass.), and the U6 promoter and FANCC-specific gRNA were synthesized as a G-block (Integrated DNA Technologies, Inc., Coralville, Iowa) and TA cloned into the pCR4 TOPO vector (Invitrogen, Carlsbad, Calif.). The right donor arm was cloned from the human genome and consisted of an 849 bp sequence. The left arm was synthesized from overlapping G-block fragments in order to introduce the corrective base and silent mutations at the TALEN and CRISPR cut sites. The donor arms flanked a floxed PGK-puromycin-T2A-FANCC cDNA selection cassette; the full donor sequence is provided as SEQ ID NO:1.

### Gene Transfer

For 293 transfections, TALENs or CRISPR/Cas9 nuclease and nickase with gRNA were delivered with LIPOFECTAMINE 2000 (Invitrogen, Carlsbad, Calif.) at a concentration of 1 μg each. Fibroblast gene transfer was performed using the Neon Transfection System (Invitrogen, Carlsbad, Calif.) using: 1500 V, 20 ms pulse width, and a single pulse. Concentrations of DNA for gene correction were: Cas9 nuclease/nickase: 1 gRNA 200 ng, and 5 μg of donor. For 48 hours after gene transfer all cells were incubated at 31° C.

### SURVEYOR Nuclease

Genomic DNA was isolated from 293 cells at 72 hours post-TALEN or CRISPR gene transfer and was amplified for 30 cycles with FANCC Forward (5′-AGACCACCCCCATGTACAAA-3′, SEQ ID NO:2) and FANCC Reverse (5′-GGAAAACCCTTCCTGGTTTC-3′, SEQ ID NO:3). It was then subjected to SURVEYOR nuclease (Transgenomic, Inc., Omaha, Nebr.) treatment as previously described (Guschin et al., 2010, Methods in molecular biology 649:247-256). Cleavage products were subjected to 10% TBE PAGE gel resolution (Invitrogen, Carlsbad, Calif.), and Image J (Research Service Branch, National Institute of Mental Health, Bethesda, Md.) was used to perform densitometry.

Gel images were utilized to determine rates of cleavage using the following equation: % gene modification=100×(1−(1−fraction cleaved)½) (Guschin et al., 2010, Methods in Molecular Biology 649:247-256). The fraction cleaved is determined using Image J and is the densitometric value of the cleavage products divided by the total densitometric value for all of the peaks. The exposure times for the gels in FIG. 1(F), FIG. 1(G), and FIG. 9 were 750 milliseconds and 1.5 seconds, and 3 seconds, respectively.

### Traffic Light Reporter Cell Line Generation and Testing.

The PGE-200 pRRL TLR2.1 sEF1a Puro WPRE parental plasmid was digested with SbfI and SpeI for ligation of the following oligonucleotides that inserted the FANCC CRISPR target site into the interrupted GFP portion of the plasmid: 5′-GGCACCTATAGATTACTATCCTGGA-3′ (SEQ ID NO:21) and 5′-CTAGTCCAGGATAGTAATCTATAGGTGCCTGCA-3′ (SEQ ID NO:22). Lentiviral particles were prepared by packaging with Addgene plasmids: 12259 (pMD2.G) 12251(pMDLg/pRRE), and 12253 (pRSV-Rev) (Addgene, Cambridge, Mass.) in 293T cells transfected with LIPOFECTAMINE 2000 (Invitrogen, Carlsbad, Calif.). The cell culture volume for viral production was 20 mL and viral particles were collected for 48 hours and 20 μl of the supernatant was added to 293T cells followed by puromycin selection with 0.3 μg/mL. This reporter line was transfected with 1 μg each of the Cas9 nuclease or nickases and 1 μg of the gRNA with the indicated concentrations of the pCVL SFFV d14GFP Donor (Addgene 31475, Addgene, Cambridge, Mass.). Green or red fluorescence was analyzed 72 hours post transfection using the BD LSRFortessa™ Cell Analyzer (BD Biosciences, San Jose, Calif.).

### Selection and Transgenic Excision

Seven days after gene transfer, cells were selected in bulk in 0.2 mg/mL puromycin. Resistant cells were then plated at low density (˜500 cells in a 10 cm² dish) for three days followed by silicone grease-coated cloning disk placement (Corning, Inc., Corning, N.Y.). Isolated colonies were progressively passed to larger culture vessels so that cell culture confluency was maintained between 50%-70% under hypoxic culture conditions.

Cells confirmed to have undergone HDR were seeded into 24-well plates and serially transfected with a CAGGs promoter driven Cre-recombinase (Addgene: 13775, Addgene, Cambridge, Mass.) or transduced with an adenoviral cre (Vector Biolabs, Malvern, Pa.) at an MOI of 10. Excision was confirmed using primers: FC CRP 491 F (5′-GAAACCAGGAAGGGTTTTCC-3′, SEQ ID NO:23) and FC CRP 1163R (5′-CAACCCCCATCTTCTCATGT-3′, SEQ ID NO:24).

### Cell Correction Molecular Screening

Primer pairs were designed to amplify a junction between the donor right arm and endogenous locus using donor-specific forward: (5′-GCCACTCCCACTGTCCTTTCCT-3′, SEQ ID NO:4) and FANCC reverse (5′-ccaagtccctcagtcccaga-3′, SEQ ID NO:5). To confirm homology-directed repair on the left portion of the correction template, the FANCC genomic Forward (5′-CAGACACACCCCTGGAAGTC-3′, SEQ ID NO:6) and donor reverse (5′-CTTTTGAAGCGTGCAGAATGCC-3′, SEQ ID NO:7). For RT-PCR, total cellular RNA was isolated and reverse transcribed using SuperScript Vilo (Invitrogen, Carlsbad, Calif.) followed by amplification with: FANCC allele-specific RT forward (5′-GGTGTATTAAGCCATATTCTGAGC-3′, SEQ ID NO:8) and reverse (5′-ACAACCCGGAATATGGCAGG-3′, SEQ ID NO:9). PCR products were cloned into the pCR 4 TOPO vector (Invitrogen, Carlsbad, Calif.) for Sanger sequencing confirmation of the entire amplicon using the M13 forward and reverse primers.

H2AX staining was performed on cells seeded at a concentration of 120,000 total cells in a T25 flask in the presence of 2 mM hydroxyurea (Sigma-Aldrich, St. Louis, Mo.) for 48 hours using the H2AX phosphorylation assay kit according to the manufacturers instructions (EMD Millipore, Billerica, Mass.). Flow cytometry was performed using the BD LSRFortessa™ Cell Analyzer (BD Biosciences, San Jose, Calif.).

### Off Target Analysis

TALEN or CRISPR/Cas9 nuclease/nickase and gRNA plasmids (1 μg each) were delivered to 293 cells by lipofection. These cells were used for SURVEYOR analysis or gene tagging with integrase-deficient lentiviral (IDLV). The p11CMV-GFP expression vector, the pCMV-AR8.2 packaging plasmid harboring the D64V integrase mutation (Lombardo et al., 2007, Nature biotechnology 25:1298-1306), and the pMD2.VSV-G envelope-encoding plasmid (Addgene 12259, Addgene, Cambridge, Mass.) were delivered to the 293T viral producing line with LIPOFECTAMINE 2000 (Invitrogen, Carlsbad, Calif.). Addition of GFP IDLV at an MOI of 5 occurred 24 hours post-nuclease delivery.

Seven days post IDLV addition, the cells were sorted for GFP and then expanded. SURVEYOR analysis was performed with the FANCC primers listed above and OT1 (F: 5′-TGGGTGGAGGTAGTTTCCTG-3′ (SEQ ID NO:10) and R: 3′-AGTGGGAAGAGGGCTGATTT-3′ (SEQ ID NO:11)), OT2 (F: 5′-TCTGGGCATAAAGAAGGTGTG-3′ (SEQ ID NO:12) and R: 5′-ATTGACTCATCTCGGGCATT-3′ (SEQ ID NO:13)), OT3 (F: 5′-GACCTGGGCTTGAATGTGTT-3′ (SEQ ID NO:14) and R: 5′-GCAGTTGCTGTAGAATAGGCTGT-3′ (SEQ ID NO:15)), OT4 (F: 5′-CCCAGAGCAAAACCATTCAT-3′ (SEQ ID NO:16) and R: 5′-CACCTGTTGCAGACTCCTCA-3′ (SEQ ID NO:17)), and OT5 (F: 5′-AGGAGCTGGGACACTGCTAA-3′ (SEQ ID NO:18) and R: 5′-ACACATGCCTGTCCTTCTCC-3′ (SEQ ID NO:19)). These amplicons were subjected to SURVEYOR analysis as described above.

IDLV;FANCC or off-target detection PCR was performed with the LTR forward primer (5′-GTGTGACTCTGGTAACTAGAG-3′ (SEQ ID NO:20)) and the corresponding FANCC or off-target reverse primers from above. IDLV:FANCC junction amplicons were cloned and Sanger sequenced.

### Genome Wide Screening

Duplicate samples underwent nrLAM PCR or LAM PCR with MseI or MluCI as previously described (Ramirez et al., 2012, Nucleic Acids Res. 40(12):5560-5568; Ran et al., 2013, Cell 154(6):1380-1389) except that these deep sequencing data were generated with the Illumina MiSeq platform (San Diego, Calif.). Data set analysis, vector trimming, genome alignment, and IS/CLIS identification was determined using the high-throughput insertion site analysis pipeline (Arens et al., 2012, Hum Gene Ther Methods 23(2):111-118).

### Human CD34 Culture, Isolation, and Gene Transfer

Umbilical cord blood (UCB) was collected in accordance with the University of Minnesota Institutional Review Board requirements for research on human subjects. Total UCB was placed in IMDM expansion media with 100 ng/mL of IL-3, 11-6, GM-SCF, Flt-31, and stem cell factor with 1× penicillin/streptomycin and 10% human plasma and 1 μM SR1 aryl hydrocarbon receptor antagonist. CD34 cells were isolated using the EASYSEP Human CD34 Positive Selection Kit according to the manufacturer's instructions (Stemcell Technologies, Inc., Vancouver, BC) and placed back in expansion media overnight. Gene transfer was performed using the Neon Electroporator (Invitrogen, Carlsbad, Calif.) with settings of: 1400V, 10 ms pulse, with three pulses. Dose of DNA was: 1 μg GFP and 1 μg each of Cas9 (nuclease and nickases) and gRNA. 72 hours after transfection the genomic DNA was harvested for FANCC locus SURVEYOR analysis as above.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The preceding disclosure is not limited to particular aspects or embodiments described, as such may, of course, vary. Also, the terminology used herein is for the purpose of describing particular aspects only, and is not intended to be limiting, since the scope of the present disclosure will be limited only by the appended claims.

### Sequence Listing Free Text

### FANCC Donor Sequence (SEQ ID NO:1)

The left arm is indicated in bold; the right arm is indicated in bold and underlined; the floxed PGK-puromycin-T2A-FANCC cDNA selection cassette is indicated in italics; within the selection cassette, the FANCC sequence is underlined.

CAGCCAAGCCTCTTCCCTGATGATTTACTCCCAGGATTTTCAGTTCT
CAGAGTTCTTTCTCATTCAGATACTTGAAAAATGTTCATGTTTTCTT
CTTGTGTATTATTTCATTTTTACATTTATCTCTTTGATTTACATTAA
ACTGCAGTATATTTTGATGAATAATACCCGGTGAGAATCTTTTTTTC
TTTTTACAGATAGTTTTTGAGGCACCATTTATTACATAATCTGTCCT
TTCCCTACTGATTTGTGATGTGTTCTCTATCTTATATTAAATTCTAA
TATCTGGATCCTTTTGTAGTTCATGAGCGTGATGATTGGGTGTTTCA
CGCATGTGTGTGCAATGTGCCACCCTTGAACCTTGTATGACATCGGC
ACGTTACCCATCTGACCTCAAAAAAAAAGCAAAGAAAAATTATCATC
TCTGTTAAACATATTTTACTGAGTTTAAAAACAATAAAGATTCCATT
CTTAATATAGGTAAAGCACTGCTCATTGATGATATATATTTTTGTTT
CACTGCTAATGTTTGTTTAAATTGACTTCTTTTTAATGTGTTAACTT
TAATGCTAACATTTCTCTTTTACACTTTGAATCAAAGTAAATTGGGT
ACTTTGACAAACAGATTTTTTTGTTTCATAGAGACCACCCCCATCTA
CAAATAAATTGTAGGCATTGTACATAAAAGGCACTTGCATTTACTTT
TAAAGAAGTTAACTTTTTCTGTTTATGTTTTTTAGGGTGTATTAAGC
CATATTCTGAGCGCGCTGCGCTTTGATAAAGAAGTGGCGCTGTTTAC
CCAGGGCCTGGGCTATGCGCCGATTGATTATTATCCGGGCCTGCTGA
AAAATGTGAGTATTTAAAATTTATCACTTTTGAAATGTTTAATGCTG
AATGTGCCATCAGCAAAAAGAGTAAATGGAAATATTTCAGTCCTCCA
GAAGAGATGTTTAACTTTTCTTTGTTTATCTCTTCTTACCTTGGGCA
GACTTATGGCCATGTACGGAAGAAATGTGAGATGGGAAGTTATGAGA
AAGAAGGAAACCAGGAAGGGTTTTCCTAAACCAACCAATCAGCCTCT
CTTTCTAGGGACACATCTCACTTATTCACTCAGAGATGTTTGGGAGA
AGAGCTGTTCTTAGCTATTATAAACACAGTCTTGTACTGTTGAAAGA
ATCTTGTATTTCAAATAACCTGATTGGAATTTTCTGTTAAAGCAAAA
CAGAAAATTCAGTACATAGTTTTAAATATTTACCTCTTAATATTAAA
GCATTGTTTTCTTCATAACTTCGTATAATGTATGCTATACGAAGTTA
TCAAGGCAGTCTGGAGCATGCGCTTTAGCAGCCCCGCTGGGCACTTG
GCGCTACACAAGTGGCCTCTGGCCTCGCACACATTCCACATCCACCG
GTAGGCGCCAACCGGCTCCGTTCTTTGGTGGCCCCTTCGCGCCACCT
TCTACTCCTCCCCTAGTCAGGAAGTTCCCCCCCGCCCCGCAGCTCGC
GTCGTGCAGGACGTGACAAATGGAAGTAGCACGTCTCACTAGTCTCG
TGCAGATGGACAGCACCGCTGAGCAATGGAAGCGGGTAGGCCTTTGG
GGCAGCGGCCAATAGCAGCTTTGCTCCTTCGCTTTCTGGGCTCAGAG
GCTGGGAAGGGGTGGGTCCGGGGGCGGGCTCAGGGGCGGGCTCAGGG
GCGGGGCGGGCGCCCGAAGGTCCTCCGGAGGCCCGGCATTCTGCACG
CTTCAAAAGCGCACGTCTGCCGCGCTGTTCTCCTCTTCCTCATCTCC
GGGCCTTTCGACCTGCAGCCCAAGCTTACCATGACCGAGTACAAGCC
CACGGTGCGCCTCGCCACCCGCGACGACGTCCCCAGGGCCGTACGCA
CCCTCGCCGCCGCGTTCGCCGACTACCCCGCCACGCGCCACACCGTC
GATCCGGACCGCCACATCGAGCGGGTCACCGAGCTGCAAGAACTCTT
CCTCACGCGCGTCGGGCTCGACATCGGCAAGGTGTGGGTCGCGGACG
ACGGCGCCGCGGTGGCGGTCTGGACCACGCCGGAGAGCGTCGAAGCG
GGGGCGGTGTTCGCCGAGATCGGCCCGCGCATGGCCGAGTTGAGCGG
TTCCCGGCTGGCCGCGCAGCAACAGATGGAAGGCCTCCTGGCGCCGC
ACCGGCCCAAGGAGCCCGCGTGGTTCCTGGCCACCGTCGGCGTCTCG
CCCGACCACCAGGGCAAGGGTCTGGGCAGCGCCGTCGTGCTCCCCGG
AGTGGAGGCGGCCGAGCGCGCCGGGGTGCCCGCCTTCCTGGAGACCT
CCGCGCCCCGCAACCTCCCCTTCTACGAGCGGCTCGGCTTCACCGTC
ACCGCCGACGTCGAGGTGCCCGAAGGACCGCGCACCTGGTGCATGAC
CCGCAAGCCCGGTGCCGAGGGCAGAGGAAGTCTGCTAACATGCGGTG
ACGTCGAGGAGAATCCTGGCCCAGCTCAAGATTCAGTAGATCTTTCT
TGTGATTATCAGTTTTGGATGCAGAAGCTTTCTGTATGGGATCAGGC
TTCCACTTTGGAAACCCAGCAAGACACCTGTCTTCACGTGGCTCAGT
TCCAGGAGTTCCTAAGGAAGATGTATGAAGCCTTGAAAGAGATGGAT
TCTAATACAGTCATTGAAAGATTCCCCACAATTGGTCAACTGTTGGC
AAAAGCTTGTTGGAATCCTTTTATTTTAGCATATGATGAAAGCCAAA
AAATTCTAATATGGTGCTTATGTTGTCTAATTAACAAAGAACCACAG
AATTCTGGACAATCAAAACTTAACTCCTGGATACAGGGTGTATTATC
TCATATACTTTCAGCACTCAGATTTGATAAAGAAGTTGCTCTTTTCA
CTCAAGGTCTTGGGTATGCACCTATAGATTACTATCCTGGTTTGCTT
AAAAATATGGTTTTATCATTAGCGTCTGAACTCAGAGAGAATCATCT
TAATGGATTTAACACTCAAAGGCGAATGGCTCCCGAGCGAGTGGCGT
CCCTGTCACGAGTTTGTGTCCCACTTATTACCCTGACAGATGTTGAC
CCCCTGGTGGAGGCTCTCCTCATCTGTCATGGACGTGAACCTCAGGA
AATCCTCCAGCCAGAGTTCTTTGAGGCTGTAAACGAGGCCATTTTGC
TGAAGAAGATTTCTCTCCCCATGTCAGCTGTAGTCTGCCTCTGGCTT
CGGCACCTTCCCAGCCTTGAAAAAGCAATGCTGCATCTTTTTGAAAA
GCTAATCTCCAGTGAGAGAAATTGTCTGAGAAGGATCGAATGCTTTA
TAAAAGATTCATCGCTGCCTCAAGCAGCCTGCCACCCTGCCATATTC
CGGGTTGTTGATGAGATGTTCAGGTGTGCACTCCTGGAAACCGATGG
GGCCCTGGAAATCATAGCCACTATTCAGGTGTTTACGCAGTGCTTTG
TAGAAGCTCTGGAGAAAGCAAGCAAGCAGCTGCGGTTTGCACTCAAG
ACCTACTTTCCTTACACTTCTCCATCTCTTGCCATGGTGCTGCTGCA
AGACCCTCAAGATATCCCTCGGGGACACTGGCTCCAGACACTGAAGC
ATATTTCTGAACTGCTCAGAGAAGCAGTTGAAGACCAGACTCATGGG
TCCTGCGGAGGTCCCTTTGAGAGCTGGTTCCTGTTCATTCACTTCGG
AGGATGGGCTGAGATGGTGGCAGAGCAATTACTGATGTCGGCAGCCG
AACCCCCCACGGCCCTGCTGTGGCTCTTGGCCTTCTACTACGGCCCC
CGTGATGGGAGGCAGCAGAGAGCACAGACTATGGTCCAGGTGAAGGC
CGTGCTGGGCCACCTCCTGGCAATGTCCAGAAGCAGCAGCCTCTCAG
CCCAGGACCTGCAGACGGTAGCAGGACAGGGCACAGACACAGACCTC
AGAGCTCCTGCACAACAGCTGATCAGGCACCTTCTCCTCAACTTCCT
GCTCTGGGCTCCTGGAGGCCACACGATCGCCTGGGATGTCATCACCC
TGATGGCTCACACTGCTGAGATAACTCACGAGATCATTGGCTTTCTT
GACCAGACCTTGTACAGATGGAATCGTCTTGGCATTGAAAGCCCTAG
ATCAGAAAAACTGGCCCGAGAGCTCCTTAAAGAGCTGCGAACTCAAG
TCTAGCATCATCACCATCACCATTGAGTTTAAACCCGCTGATCAGCC
TCGACTGTGCCTTCTAGTTGCCAGCCATCTGTTGTTTGCCCCTCCCC
CGTGCCTTCCTTGACCCTGGAAGGTGCCACTCCCACTGTCCTTTCCT
AATAAAATGAGGAAATTGCATCGCATTGTCTGAGTAGGTGTCATTCT
ATTCTGGGGGGTGGGGTGGGGCAGGACAGCAAGGGGGAGGATTGGGA
AGACAATAGCAGGCATGCTGGGGATGCGGTGGGCTCTATGGCTTCTG
AGGCGGAAAGAACCAGCTATAACTTCGTATAATGTATGCTATACGAA
GTTATTGGCTTAAATTATAGCCAAATGTGAGAAATTTTAACTTACAA
ACTTGATGTAACTCTTCTAAAAAATGTTATGAGATTTTTTAATGTTT
GTTAACCATTCCGGTGTTTTGGAAGCTTTGTACAATAACAACTTTTT
TTTTTTTTTCAAATGAACTGAATTTTAAATGAAGAAGTAAACTAACT
TTTCTTTAAATGGATTTGGTTTTAATTCTTAGGAAATTAATGACCTG
TCTATTGTTCATTGCTTAAATAGGAATGCAGAATTATAGACATTAAA
CATAAAATCCCAATTATTAGTAAATGTGACATGGCACTGCTTCCTTT
TCACTCTGACAGAGTGAAACATGAGAAGATGGGGGTTGGGGGAATCT
CAACGGAAATATCACTCACACCAAGAAAAATAGAACTGATGTAATCC
TGTTTGCAGCGTGAGTTAACCTGCAACTGATTTTGTTTTACAGATGG
TTTTATCATTAGCGTCTGAACTCAGAGAGAATCATCTTAATGGATTT
AACACTCAAAGGCGGTAGGTGTTAAACTAAACATCCTTCTTCTCAGG
TTTCAAAATGTATCAGTTTGGTTATGAGAGGAAAATTTTACAATTCA
TAGGAAATGGATGTTCAGTTATGGTTGTATTTTATATAGAAAGATTA
TTTTAGTGGAATACATAGCAAATTGGTGAGTTTTTTCAAACTCTTTT
AAAAATCACTATTTTCTCAACTCTCACAGAGCAGTAAGTAAATCATA
CAATGTCTTTTGTGGGCCCATTAGGTAGAAAGCCCTACATACACAGT
AGGGAGCATAAAAGAAAGAGCAGTATTAGTTTTACCCTGGGATTGCT
CACTCTG

1. A method of changing a genomic sequence, the method comprising:
introducing into a cell that comprises a genomic sequence in need of editing:
a donor polynucleotide that encodes an edited version of the sequence in need of editing; and
a polynucleotide that encodes an enzyme that cuts at least one strand of DNA;
allowing the enzyme to cut at least one strand of the genomic sequence; and
allowing the donor sequence to replace the genomic sequence in need of editing.

2. The method of claim 1, wherein the genomic sequence comprises a FANCC locus with a c.456+4A>T mutation or an equivalent thereof.

3. The method of claim 1, wherein the donor polynucleotide comprises a FANCC locus with a wild-type c.456+4A or an equivalent thereof.

4. The method of claim 3, wherein the edited version of the sequence in need of editing comprises the FANCC locus with a wild-type c.456+4A or an equivalent thereof.

5. The method of claim 1 wherein the enzyme comprises a nuclease.

6. The method of claim 5 wherein the nuclease comprises FokI or Cas9 nuclease.

7. The method of claim 5 wherein the enzyme comprises a nickase.

8. The method of claim 7 wherein the nickase comprises RuvC of Cas9.

9. The method of claim 1 wherein the donor polynucleotide further comprises a selectable marker.

10. The method of claim 1 wherein the donor polynucleotide further comprises at least one silent DNA polymorphism.

11. The method of claim 1 wherein the donor sequence replaces the genomic sequence in need of editing by homology-directed repair.

12. The method of claim 1 wherein the donor sequence replaces the genomic sequence in need of editing by non-homologous end-joining.

13. The method of claim 1, wherein the cell is a pluripotent cell, a multipotent cell, a differentiated cell, or a stem cell.

14. The method of claim 13, wherein the cell is homozygous for the c.456+4A>T mutation.

15. The method of claim 13, wherein the stem cell is a CD34+ human hematopoietic stem cell.

16. The method of claim 1, wherein the stem cell is a mammalian stem cell.

17. The method of claim 16 wherein the mammalian stem cell comprises a human stem cell or a murine stem cell.

18. An isolated cell prepared by the method of claim 1.

19. A population of cells of claim 18.

20. An expanded population of cells of claim 19.

21. A composition comprising the cell of claim 18 and a carrier.

22. The composition of claim 21, wherein the carrier is a pharmaceutically acceptable carrier.

23. A polynucleotide comprising:
a promoter sequence;
a polynucleotide encoding a functional portion of a Cas9 nuclease operably linked to the promoter sequence; and
a polyadenylation signal operably linked to the polynucleotide encoding a functional portion of a Cas9 nuclease.

24. A polynucleotide comprising:
a promoter sequence;
a polynucleotide encoding a functional portion of a Cas9 nickase operably linked to the promoter sequence; and
a polyadenylation signal operably linked to the polynucleotide encoding a functional portion of a Cas9 nickase.

25. A method of treating a condition in a subject caused by a genetic mutation, the method comprising:
obtaining a plurality of pluripotent cells from the subject, the pluripotent cells comprising a genomic sequence that comprises the genetic mutation;
introducing into at least one cell:
a donor polynucleotide that encodes a version of the genomic sequence edited with respect to the genetic mutation; and
a polynucleotide that encodes an enzyme that cuts at least one strand of DNA;
allowing the enzyme to cut at least one strand of the genomic sequence;
allowing the donor sequence to replace the genomic sequence that comprises the genetic mutation with the edited version, thereby producing an edited genomic sequence;
expanding the cell that comprises the edited genomic sequence; and
introducing a plurality of the expanded cells comprising the edited genomic sequence into the subject.

26. The method of claim 25 wherein the condition comprises Fanconi's anemia.

27. The method of claim 26 wherein the genomic sequence that comprises a genetic mutation comprises a FANCC locus with a wild-type c.456+4A>T mutation.

28. The method of claim 27 wherein the donor polynucleotide encodes a FANCC locus with a wild-type c.456+4A.

29. The method of claim 26 wherein introducing the plurality of expanded cells into the subject results in correction of the FANCC locus.

30. The method of claim 26 wherein introducing the plurality of expanded cells into the subject results in restoration of proper splicing of FANCC mRNA.

31. The method of claim 26 wherein introducing the plurality of expanded cells into the subject results in phenotypic rescue of the subject.

32. A composition comprising the population of claim 19, and a carrier.

33. A composition comprising the population of claim 20, and a carrier.

34. The method of claim 27 wherein introducing the plurality of expanded cells into the subject results in correction of the FANCC locus.

35. The method of claim 27 wherein introducing the plurality of expanded cells into the subject results in restoration of proper splicing of FANCC mRNA.

36. The method of claim 27 wherein introducing the plurality of expanded cells into the subject results in phenotypic rescue of the subject.

37. The method of claim 28 wherein introducing the plurality of expanded cells into the subject results in correction of the FANCC locus.

38. The method of claim 28 wherein introducing the plurality of expanded cells into the subject results in restoration of proper splicing of FANCC mRNA.

39. The method of claim 28 wherein introducing the plurality of expanded cells into the subject results in phenotypic rescue of the subject.


SEQUENCE LISTING (56 sequences)

SEQ ID NO: 1 - DNA, 5317 bp, ARTIFICIAL
  donor sequence
cagccaagcctcttccctgatgatttactcccaggattttcagttctcagagttctttctcattcagatacttgaaaaatgttcatgttttcttcttgtgtattatttcatttttacatttatctctttgatttacattaaactgcagtatattttgatgaataatacccggtgagaatctttttttctttttacagatagtttttgaggcaccatttattacataatctgtcctttccctactgatttgtgatgtgttctctatcttatattaaattctaatatctggatccttttgtagttcatgagcgtgatgattgggtgtttcacgcatgtgtgtgcaatgtgccacccttgaaccttgtatgacatcggcacgttacccatctgacctcaaaaaaaaagcaaagaaaaattatcatctctgttaaacatattttactgagtttaaaaacaataaagattccattcttaatataggtaaagcactgctcattgatgatatatatttttgtttcactgctaatgtttgtttaaattgacttctttttaatgtgttaactttaatgctaacatttctcttttacactttgaatcaaagtaaattgggtactttgacaaacagatttttttgtttcatagagaccacccccatctacaaataaattgtaggcattgtacataaaaggcacttgcatttacttttaaagaagttaactttttctgtttatgttttttagggtgtattaagccatattctgagcgcgctgcgctttgataaagaagtggcgctgtttacccagggcctgggctatgcgccgattgattattatccgggcctgctgaaaaatgtgagtatttaaaatttatcacttttgaaatgtttaatgctgaatgtgccatcagcaaaaagagtaaatggaaatatttcagtcctccagaagagatgtttaacttttctttgtttatctcttcttaccttgggcagacttatggccatgtacggaagaaatgtgagatgggaagttatgagaaagaaggaaaccaggaagggttttcctaaaccaaccaatcagcctctctttctagggacacatctcacttattcactcagagatgtttgggagaagagctgttcttagctattataaacacagtcttgtactgttgaaagaatcttgtatttcaaataacctgattggaattttctgttaaagcaaaacagaaaattcagtacatagttttaaatatttacctcttaatattaaagcattgttttcttcataacttcgtataatgtatgctatacgaagttatcaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgagggcagaggaagtctgctaacatgcggtgacgtcgaggagaatcctggcccagctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatggtccaggtgaaggccgtgctgggccacctcctggcaatgtccagaagcagcagcctctcagcccaggacctgcagacggtagcaggacagggcacagacacagacctcagagctcctgcacaacagctgatcaggcaccttctcctcaacttcctgctctgggctcctggaggccacacgatcgcctgggatgtcatcaccctgatggctcacactgctgagataactcacgagatcattggctttcttgaccagaccttgtacagatggaatcgtcttggcattgaaagccctagatcagaaaaactggcccgagagctccttaaagagctgcgaactcaagtctagcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctataacttcgtataatgtatgctatacgaagttattggcttaaattatagccaaatgtgagaaattttaacttacaaacttgatgtaactcttctaaaaaatgttatgagattttttaatgtttgttaaccattccggtgttttggaagctttgtacaataacaacttttttttttttttcaaatgaactgaattttaaatgaagaagtaaactaacttttctttaaatggatttggttttaattcttaggaaattaatgacctgtctattgttcattgcttaaataggaatgcagaattatagacattaaacataaaatcccaattattagtaaatgtgacatggcactgcttccttttcactctgacagagtgaaacatgagaagatgggggttgggggaatctcaacggaaatatcactcacaccaagaaaaatagaactgatgtaatcctgtttgcagcgtgagttaacctgcaactgattttgttttacagatggttttatcattagcgtctgaactcagagagatcatcttaatggatttaacactcaaaggcggtaggtgttaaactaaacatccttcttctcaggtttcaaaatgtatcagtttggttatgagaggaaaattttacaattcataggaaatggatgttcagttatggttgtattttatatagaaagattattttagtggaatacatagcaaattggtgagttttttcaaactcttttaaaaatcactattttctcaactctcacagagcagtaagtaaatcatacaatgtcttttgtgggcccattaggtagaaagccctacatacacagtagggagcataaaagaaagagcagtattagttttaccctgggattgctcactctg

SEQ ID NO: 2 - DNA, 20 bp, Artificial
  Primer
agaccacccccatgtacaaa

SEQ ID NO: 3 - DNA, 20 bp, Artificial
  Primer
ggaaaacccttcctggtttc

SEQ ID NO: 4 - DNA, 22 bp, ARTIFICIAL
  Primer
gccactcccactgtcctttcct

SEQ ID NO: 5 - DNA, 20 bp, ARTIFICIAL
  Primer
ccaagtccctcagtcccaga

SEQ ID NO: 6 - DNA, 20 bp, ARTIFICIAL
  Primer
cagacacacccctggaagtc

SEQ ID NO: 7 - DNA, 22 bp, ARTIFICIAL
  Primer
cttttgaagcgtgcagaatgcc

SEQ ID NO: 8 - DNA, 24 bp, ARTIFICIAL
  Primer
ggtgtattaagccatattctgagc

SEQ ID NO: 9 - DNA, 20 bp, ARTIFICIAL
  Primer
acaacccggaatatggcagg

SEQ ID NO: 10 - DNA, 20 bp, ARTIFICIAL
  Primer
tgggtggaggtagtttcctg

SEQ ID NO: 11 - DNA, 20 bp, ARTIFICIAL
  Primer
agtgggaagagggctgattt

SEQ ID NO: 12 - DNA, 21 bp, ARTIFICIAL
  Primer
tctgggcataaagaaggtgtg

SEQ ID NO: 13 - DNA, 20 bp, ARTIFICIAL
  Primer
attgactcatctcgggcatt

SEQ ID NO: 14 - DNA, 20 bp, ARTIFICIAL
  Primer
gacctgggcttgaatgtgtt

SEQ ID NO: 15 - DNA, 23 bp, ARTIFICIAL
  Primer
gcagttgctgtagaataggctgt

SEQ ID NO: 16 - DNA, 20 bp, ARTIFICIAL
  Primer
cccagagcaaaaccattcat

SEQ ID NO: 17 - DNA, 20 bp, ARTIFICIAL
  Primer
cacctgttgcagactcctca

SEQ ID NO: 18 - DNA, 20 bp, ARTIFICIAL
  Primer
aggagctgggacactgctaa

SEQ ID NO: 19 - DNA, 20 bp, ARTIFICIAL
  Primer
acacatgcctgtccttctcc

SEQ ID NO: 20 - DNA, 21 bp, ARTIFICIAL
  Primer
gtgtgactctggtaactagag

SEQ ID NO: 21 - DNA, 25 bp, ARTIFICIAL
  Primer
ggcacctatagattactatcctgga

SEQ ID NO: 22 - DNA, 33 bp, ARTIFICIAL
  Primer
ctagtccaggatagtaatctataggtgcctgca

SEQ ID NO: 23 - DNA, 20 bp, ARTIFICIAL
  Primer
gaaaccaggaagggttttcc

SEQ ID NO: 24 - DNA, 20 bp, ARTIFICIAL
  Primer
caacccccatcttctcatgt

SEQ ID NO: 25 - DNA, 105 bp, Homo Sapiens
tctcatatactttcagcactcagatttgataaagaagttgctcttttcactcaaggtcttgggtatgcacctatagattactatcctggtttgcttaaaatgtgt

SEQ ID NO: 26 - DNA, 63 bp, Homo Sapiens
ctcatatactttcagcactcagatttgataaagaagttgctcttttcactcaaggtcttgggt

SEQ ID NO: 27 - DNA, 20 bp, Homo Sapiens
cgtggatatctaatgatagg

SEQ ID NO: 28 - DNA, 77 bp, Artificial
  Primer
gcacctatagattactatccgagagcagaaaagcaagaaaaaaggcagccgacaacgaaaaagggcaccgagcgggc

SEQ ID NO: 29 - DNA, 23 bp, Homo Sapiens
gcacctatagattactatcctgg

SEQ ID NO: 30 - DNA, 23 bp, Homo Sapiens
gcacatggagattactctccaag

SEQ ID NO: 31 - DNA, 23 bp, Homo Sapiens
gtatctatagattgctatgctag

SEQ ID NO: 32 - DNA, 23 bp, Homo Sapiens
ccacctatagataattctcctgg

SEQ ID NO: 33 - DNA, 23 bp, Homo Sapiens
gcacctacagttgacaatccagg

SEQ ID NO: 34 - DNA, 23 bp, Homo Sapiens
gcacctataggttattaactgag

SEQ ID NO: 35 - DNA, 29 bp, Homo Sapiens
actatcctggtttgcttaaaaatgtgtgt

SEQ ID NO: 36 - DNA, 28 bp, Homo Sapiens
ttatccgggcctgctgaaaaatgtgagt

SEQ ID NO: 37 - DNA, 25 bp, Homo Sapiens
ggtgtattaagccatattctgagcg

SEQ ID NO: 38 - DNA, 32 bp, Homo Sapiens
gaaaaatatggttttatcattagcgtctgaac

SEQ ID NO: 39 - DNA, 21 bp, ARTIFICIAL
  Primer
gtgtgactctggtaactagag

SEQ ID NO: 40 - DNA, 21 bp, ARTIFICIAL
  Primer
ggaaaacccttcctggtttcc

SEQ ID NO: 41 - DNA, 23 bp, ARTIFICIAL
  Primer
gcacctatagattactatcctgg

SEQ ID NO: 42 - DNA, 286 bp, ARTIFICIAL
  Primer
gtgtgactctggtaactagagatccctcagacccttttagtcagtgtggaaaatctcttcctggtttgcttaaaaatgtgagtatttaaaatttatcacttttgaaatgtttaatgctgaatgtgccatcagcaaaaagagtaaatggaaatatttcagtcctccagaagagatgtttaacttttctttgtttatctcttcttaccttgggcagacttatggccatgtacggaagaaatgtgagatgggaagttatgagaaagaaggaaaccaggaagggttttcc

SEQ ID NO: 43 - DNA, 344 bp, ARTIFICIAL
  Primer
gtgtgactctggtaactagagatccctcagacccttttagtaagtgtggaaaatatctagcagtattttatataccacagccaatttgttatgttaaaccaattccacaaacttgcacctggtttgcttaaaaatgtgagtatttaaaatttatcacttttgaaatgtttaatgctgaatgtgccatcagcaaaaagagtaaatggaaatatttcagtcctccagaagagatgtttaactttcctttgtttatctcttcttaccttgggcagacttatggccatgtacggaagaaatgtgagatgggaagttatgagaaagaaggaaaccaggaagggttttcc

SEQ ID NO: 44 - DNA, 285 bp, ARTIFICIAL
  Primer
gtgtgactctggtaactagagatccctcagacccttttagtcagtgtggaaaatctatcctggtttgcttaaaaatgtgagtatttaaaatttatcacttttgaaatgtttaatgctgaatgtgccatcagcaaaaagagtaaatggaaatatttcagtcctccagaagagatgtttaacttttctttgtttatctcttcttaccttgggcagacttatggccatgtacggaagaaatgtgagatgggaagttatgagaaagaaggaaaccaggaagggttttcc

SEQ ID NO: 45 - DNA, 343 bp, ARTIFICIAL
  Primer
gtgtgactctggtaactagagatccctcagacccttttagtcagtgtggaaaatctctagcagtagaagttgctcttttcactcaaggtcttgggtatgcacctatagattactatcctggtttgcttaaaaatgtgagtatttaaaatttatcacttttgaaatgtttaatgctgaatgtgccatcagcaaaaagagtaaatggaaatatttcagtcctccagaagagatgtttaacttttctttgtttatctcttcttaccttgggcagacttatggccatgtacggaagaaatgtgagatgggaagttatgagaaagaaggaaaccaggaagggttttcc

SEQ ID NO: 46 - DNA, 296 bp, ARTIFICIAL
  Primer
gtgtgactctggtaactagagatccctcttttcactcaaggtcttgggtatgcacctatagattactatcctggtttgcttaaaaatgtgagtatttaaaatttatcacttttgaaatgtttaatgctgaatgtgccatcagcaaaaagagtaaatggaaatatttcagtcctccagaagagatgtttaacttttctttgtttatctcttcttaccttgggcagacttatggccatgtacggaagaaatgtgagatgggaagttatgagaaagaaggaaaccaggaagggttttcc

SEQ ID NO: 47 - DNA, 180 bp, Homo Sapiens
gtaaacacaagattttatttttattgagcacagccaagcctcttccctgatgatttactcccaggattttcagttctcagagttctttctcattcagatacttgaaaaatgttcatgttttcttcttgtgtattatttcatttttacatttatctctttgatttacattaaactgcagta

SEQ ID NO: 48 - DNA, 180 bp, Homo Sapiens
gtaaacacaagattttatttttattgagcacagccaagcctcttccctgatgatttactcccaggattttcagttctcagagttctttctcattcagatacttgaaaaatgttcatgttttcttcttgtgtattatttcatttttacatttatctctttgatttacattaaactggagta

SEQ ID NO: 49 - DNA, 210 bp, Homo Sapiens
cccatctacaaataaattgtaggcattgtacataaaaggcacttgcatttacttttaaagaagttaactttttctgtttatgttttttagggtgtattaagccatattctgagcgcgctgcgctttgataaagaagtggcgctgtttacccagggcctgggctatgcgccgattgattattatccgggcctgctgaaaaatgtgagtatt

SEQ ID NO: 50 - DNA, 210 bp, Homo Sapiens; identical to SEQ ID NO: 49

SEQ ID NO: 51 - DNA, 405 bp, Homo Sapiens
atggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagatggttttatcattagcgtctgaactcagagagaatcatcttaatggatttaacactcaa

SEQ ID NO: 52 - DNA, 405 bp, Homo Sapiens
atggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaactaacaaagaaccacagaattctggacaatcaaaacttaactcctggatacagatggttttatcattagcgtctgaactcagagagaatcatcttaatggatttaacactcaa

SEQ ID NO: 53 - DNA, 536 bp, Homo Sapiens
ggtgtattaagccatattctgagcgcgctgcgctttgataaagaagtggcgctgtttacccagggcctgggctatgcgccgattgattattatccgggcctgctgaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgt

SEQ ID NO: 54 - DNA, 536 bp, Homo Sapiens; identical to SEQ ID NO: 53

SEQ ID NO: 55 - DNA, 87 bp, Homo Sapiens
tcttaatattaaagcattgttttcttcataacttcgtataatgtatgctatacgaagttattggcttaaattatagccaaatgtgag

SEQ ID NO: 56 - DNA, 78 bp, Homo Sapiens
ttaaatatttacctcttaatattaaagcattgttttcttctggcttaaattatagccaaatgtgagaaattttaactt